(19) United States
(12) Patent Application Publication
(10) Pub. No.: US 2026/0237470 A1
(43) Pub. Date: Aug. 13, 2026

(54) METHOD FOR DETERMINING A TARGET FORMULATION COMPRISING A TARGET BIODEGRADABILITY

(71) Applicant: BASF SE, Ludwigshafen am Rhein (DE)

(72) Inventors: Glauco Battagliarin, Ludwigshafen am Rhein (DE); Volker Settels, Ludwigshafen am Rhein (DE); Jessica Eleanor Mugleston, Stockport, Greater Manchester (GB); Andreas Kuenkel, Ludwigshafen am Rhein (DE); Sonja Schmidt, Ludwigshafen am Rhein (DE); Constanze Risse, Ludwigshafen am Rhein (DE); Jan Philipp Herrmann, Ludwigshafen am Rhein (DE); Stefano Lazzari, Ludwigshafen am Rhein (DE); Michaela Agari, Ludwigshafen am Rhein (DE)

(21) Appl. No.: 19/140,678

(22) PCT Filed: Dec. 21, 2023
(86) PCT No.: PCT/EP2023/087415

§ 371 Date: Jun. 18, 2025

(30) Foreign Application Priority Data
Dec. 21, 2022 (EP) 22215543.4
Dec. 21, 2022 (EP) 22215576.4
Dec. 21, 2022 (EP) 22215589.7
Dec. 21, 2022 (EP) 22215601.0

(51) Int. Cl. G16C 20/30; G16C 20/70

(52) U.S. Cl. CPC G16C 20/30; G16C 20/70

(57) ABSTRACT

The invention refers to a method for determining a preparation specification comprising a target biodegradability. A target biodegradability is provided indicative of a biodegradation characteristic of a formulation. A digital representation of a potential target preparation specification is provided. A habitat is provided indicative of habitat descriptor values of habitat descriptors. A model is provided based on the habitat that is adapted to determine the biodegradability of a formulation in the habitat. The biodegradability of the potential target formulation is determined based on the provided model and the digital representation. Then the determined biodegradability is compared with the target biodegradability and either i) the potential target formulation is determined as the target formulation, or ii) a new potential target preparation specification of a potential target formulation is provided and the determination of the biodegradability is repeated utilizing the new potential target preparation specification.

### FIELD OF THE INVENTION

The invention relates to a method, an apparatus and a computer program product for determining a target preparation specification indicative of a target formulation comprising a target biodegradability. Further, the invention refers to a training method, a training apparatus and a training computer program for training a data driven biodegradation model utilizable by the method, apparatus and computer program product for determining the target preparation specification. Moreover, the invention refers to a method and apparatus for providing an interface for providing the target preparation specification.

### BACKGROUND OF THE INVENTION

Generally, formulations, i.e. products comprising at least two chemical components, are widely used in industrial and/or daily use products due to their broad range of application properties. The use of formulations encompasses amongst others coatings, personal care products, washing detergents, lubricants, packaging, films and foams. However, this widely spread application leads on the other hand to a huge amount of waste containing the used formulations. Non-degradable waste is a problem when disposing in a non-designated environment. Especially, chemical build-ups due to chemicals that do not undergo a change in chemical structure in order to be fed back into the cycle is undesired. Thus, if non-biodegradable formulations are not suitably collected in intended waste stream, this can result in increased chemical contamination in the environment. Thus, there is not only a need for formulations that decompose, but also a need to take into account knowledge about the biodegradability of a formulation in early stages of a product design process. In particular, it would be advantageous if already during a production design process formulations could be predicted that provide a specific biodegradability and that are also suitable for an intended application. Thus, it would be advantageous to provide a possibility to predict a formulation that comprises a suitable biodegradability for an application in an accurate and computationally inexpensive manner.

### SUMMARY OF THE INVENTION

It is an object of the present invention to provide a method, an apparatus and a computer program product that allow to determine a preparation specification indicative of a target formulation comprising a target biodegradability that allows for an accurate determination and is computationally inexpensive. Moreover, it is further an object of the invention to provide a training method, a training apparatus and a computer program product that allow to provide a biodegradation model that is usable in the method, apparatus and computer program and that can be trained to provide a good determination accuracy by utilizing less computational resources.

In a first aspect of the present invention, a computer implemented method for determining a target preparation specification indicative of a target formulation comprising a target biodegradability is presented, wherein the method comprises a) providing a target biodegradability, wherein a biodegradability is indicative of a biodegradation characteristic of a formulation, b) providing a digital representation of a potential target preparation specification of a potential target formulation, c) providing a biodegradation habitat, wherein the biodegradation habitat is indicative of habitat descriptor values of habitat descriptors influencing a biodegradation of a formulation in the respective habitat, wherein the habitat descriptors are indicative of environmental characteristics of the habitat, d) providing a biodegradation model based on the provided biodegradation habitat, wherein the biodegradation model is adapted to determine the biodegradability of a formulation in the respective biodegradation habitat, wherein the biodegradation model is a data driven model parameterized with respect to the biodegradation habitat such that it determines a biodegradability of a formulation based on the digital representation of a formulation, e) determining the biodegradability of the potential target formulation based on the provided biodegradation model and the digital representation, and f) comparing the determined biodegradability of the potential target formulation with the target biodegradability and, based on the comparison, either i) determining the potential target formulation as the target formulation and the potential target preparation specification as the target preparation specification, or ii) providing a new potential target preparation specification of a potential target formulation and repeating the determination of the biodegradability utilizing the new potential target preparation specification of the potential target formulation.

Since the biodegradation model is specifically adapted to determine a biodegradability of a potential target formulation with respect to a specific biodegradation habitat characterized by respective habitat descriptor values influencing a biodegradability of a formulation in the respective habitat, the biodegradability of a formulation, in particular, of a potential target formulation, for the respective habitat can be determined very accurately. Moreover, since the biodegradation model has specifically been trained for one or more specific biodegradation habitats, less training data becomes necessary for the training and the biodegradation model becomes more flexible with respect to determining the biodegradation for new formulations not being part of the training data set. Thus, an accurate determination of a biodegradability of a potential target formulation that is computationally inexpensive is provided. Since the determination of the target formulation is then based on the accurate and computationally inexpensive determination of the biodegradability, the method also allows for an accurate and computationally inexpensive determination of a target preparation specification indicative of a target formulation comprising a respective target biodegradability. Furthermore, currently utilized test methods for testing a biodegradability of a formulation are extremely time consuming and can take months or years to get results, whereas the above described method allows to provide results, in particular, a potential suitable formulation, essentially immediately. Thus, not only the technical requirements for biodegradability determination can be reduced, but also the time required for designing a new biodegradable product can be considerably shortened. Moreover, providing an easy possibility for taking an accurate determination of the biodegradability already during the design process of a product into account allows to design the product such that plastic waste, in particular, in form of micro-plastic, can be avoided. In particular, it can be ensured that a formulation used in a product will biodegrade in a respectively expected environment, for example, in a marine habitat.

Development of new chemical products that are tailored to application requirements is a predominant problem in modern chemical industries. Recently, a further requirement is also raised, related to the environmental impact of the chemical product along the life cycle of the chemical product. One important aspect of the environmental impact is prevention of chemical build-ups. Chemical build-ups are an increasing problem and can be avoided if the formulation material is biodegradable. To evaluate biodegradation currently a series of standardized tests, are used. For biodegradability, a variety of tests exist with specified conditions (e.g. ISO13432, ISO14852, ISO14855, ISO17556 and OECD 301). Standardized tests often strike a balance between a time-efficient testing (shortest 14 days, longest 24 months) and real-life conditions. In fact, higher temperatures than real conditions are often used to speed up the testing time. Companies developing new plastics need to invest significant resources in self-assessing product sustainability and in certification. The overall biodegradability assessment, including laboratory spaces and equipment, becomes costly and time consuming. Thus, there is a need to early identify the biodegradability of a new material in the development process. The proposed method of determining biodegradability as disclosed herein enables a faster and more efficient way of developing new materials. In an early phase, even before preparation of the formulation, the biodegradability can be determined. This allows to determine whether the formulation is suited for market entry. This leads to a faster time to market. This also allows to reduce waste production, because the formulation does not need to be synthesized to determine biodegradability. The proposed method provides a digital twin of measuring the biodegradability of a formulation.

Further, the standard measurements and tests for a biodegradability are often time consuming, for example, include waiting times of up to several months or even years. In particular when developing new formulations for respective applications these time consuming tests can strongly limit the development process. In this context the invention allows to provide results for a new formulation instantly strongly decreasing the time after which results are available.

Moreover, due to the incredibly high number of possible, often not even fully explored formulations, potentially suitable for a specific application, today a technical product engineer, given the technical task of finding a formulation that is not only suitable for a specific application, but also fulfills respective target properties, in particular, a target biodegradability, has to formulate and test huge amounts of possible formulations, or go through huge datasets and libraries in which potential formulations are stored in order to find a respective formulation that might fit the application. Even when utilizing sophisticated design of experiment methods, still a very high number of possible formulations has to be formulated and experimentally tested. In this context the above described method allows to assist a user, for instance, a technical product engineer, to find potentially suitable formulations automatically and much faster. In particular, by utilizing the above method the user only has to prepare and test potentially suitable formulations for which it has been determined that it is very likely that they fulfill the respective target property, in particular, a target biodegradability. Accordingly, unnecessary formulation and testing of formulations can be avoided. Thus, the method allows a user to perform a technical task of finding a formulation suitable for a technical application faster and more efficient.

The method refers to a computer implemented method and can thus be performed by a general or dedicated computer adapted to perform the method, for instance, by executing a respective computer program. The method is adapted to determine, in particular, predict, a target preparation specification indicative of a target formulation comprising a target biodegradability. Generally, a preparation specification includes instructions on how a specific associated formulation can be produced. For example, a preparation specification can refer to components, starting products and/or production conditions that, if applied, lead to a preparation of the formulation in the production process. Thus, a preparation specification is associated always with the formulation that is produced when performing the preparation specification, for instance, utilizing suitable laboratory or industrial equipment. In particular, a preparation specification can also be regarded as a recipe for how to produce the associated formulation. Since the target preparation specification and the target formulation correspond to each other, i.e. the target preparation specification, when executed accordingly, produces the target formulation, in the following both terms can be utilized concurrently, for example, when a target preparation specification is determined the target formulation is also determined and vice versa.

A biodegradable formulation refers to a formulation that can be degraded by biological processes, in particular, a bio-degradable formulation can refer to a formulation that can be assimilated by bacteria and/or fungi to give environmentally friendly products, i.e. to decompose into non-polluting residuals, for example, by producing mineralized carbon and/or biomass. Generally, a biodegradability is indicative of a biodegradation characteristic of a formulation. In particular, the biodegradability refers to a measure for a degradation, i.e. decomposition of a formulation caused by biological processes, i.e. processes that include biological material, in particular, microorganisms, taking part in the degradation process. Thus, the biodegradability does not refer to purely chemical degradation processes that do not include microbial activity. The biodegradability is an intrinsic characteristic of a formulation. In this context, an intrinsic characteristic of a formulation refers to a property of the formulation that is caused by and thus reflects the nature of a formulation, i.e. its structure, composition, etc., with respect to a specific context. In particular, the biodegradability reflects the nature of the formulation when present in a specific biological active environment. The target biodegradability can refer to any quantification of the biodegradability of a formulation. For example, the target biodegradability can refer to only one value, for instance, a half-life of the formulation in a respective habitat, or can refer to more than one value, for instance, can refer to a degradation function with time of the formulation in a specific habitat. It is preferred that the target biodegradability of the formulation refers to any one of a mineralization characteristic, a biotransformation characteristic and/or a decomposition half-life of the formulation. Preferably, the target biodegradability is provided in form of a value of the percentage of biodegradation after a predetermined timeframe.

Biodegradable formulation may be designed to degrade upon disposal by the action of living organisms. Biodegradability may relate to the environmental fate and/or behavior of the formulation. Biodegradability may relate to the extent to which the formulation can be decomposed by microorganisms such as such as bacteria, fungi or algae. Biodegradability may be dependent on the formulation's components composition of chemical structure, molecular weight, physical factors such as cross-linking density, branching, crystallinity or solubility, and exposure conditions such as habitat like soil, compost or aquatic system. With respect to exposure conditions the microorganisms, microbial population, nutrient concentration, temperature, pH, pO2, ionic condition, or substrate characteristics such as toxicity influence biodegradability. Biodegradability may be measured based on measured mass loss (mg/time), dissolved organic carbon (DOC, organic carbon concentration/time), oxygen consumption (e.g. though pressure measurement, e.g. Pa/time) or carbon dioxide production over time (e.g. though pressure measurement, e.g. Pa/time).

To quantify biodegradability in the sense of a measured property of the formulation many measurement standards have been developed. Different measurement methods are defined to determine biodegradability under pre-defined laboratory conditions. For example, for wastewater OECD Test No. 301:“Ready Biodegradability” (Jul. 17, 1992) describes 6 methods for determination of biodegradability. Further for example, ASTM D5988-18 “standard test method for determining aerobic biodegradation of plastic materials in soil” describes the measuring of the carbon dioxide developed by microorganisms as a function of time of exposure, thus measuring the degree of biodegradability relative to a reference material. Further for example ISO 17556:2019 “plastics—determination of the ultimate aerobic biodegradability of plastic materials in soil by monitoring the oxygen demand in a respirometer or the amount of carbon dioxide evolved” yields the optimum rate of biodegradation of plastic material in a test soil by controlling the oxygen consumption or the carbon dioxide production. Further for example, ISO 14855-1:2012 “determination of the ultimate aerobic biodegradability of plastic materials under controlled composting conditions—method by analysis of evolved carbon dioxide—Part 1: General method” and ASTM D5338-15 “standard test method for determining aerobic biodegradation of plastic materials under controlled composting conditions, incorporating thermophilic temperatures” determine the ultimate aerobic biodegradability (means by which microorganisms entirely consume a chemical or organic substance in the presence of oxygen) of plastics based on organic compounds under controlled composting conditions by measuring the percentage conversion of the carbon into carbon dioxide and the degree of disintegration of the plastic at the end of the test. ASTM D6400-21 “standard specification for labeling of plastics designed to be aerobically composted in municipal or industrial facilities” additionally includes elemental analysis, plant germination (phytotoxicity), and mesh filtration of the resulting particles. In ISO 17088:2021 “plastics—organic recycling—specifications for compostable plastics” includes the evaluation of negative consequences on the composting process and facility and negative effects on the quality of the resulting compost, including the presence of high levels of regulated metals and other harmful components.

For aerobic biodegradation ISO 18830:2016 “plastics—determination of aerobic biodegradation of non-floating plastic materials in a seawater/sandy sediment interface—method by measuring the oxygen demand in closed respirometer”, ISO 19679:2020 “plastics—determination of aerobic biodegradation of non-floating plastic materials in a seawater/sediment interface-method by analysis of evolved carbon dioxide” were developed. The biodegradation evaluation is measured by the oxygen demand or the CO2 evolution. Further standards for example include ISO 14853:2016 “plastics—determination of the ultimate anaerobic biodegradation of plastic materials in an aqueous system-method by measurement of biogas production”, ISO 23977-1:2020 “plastics—determination of the aerobic biodegradation of plastic materials exposed to seawater—Part 1: method by analysis of evolved carbon dioxide” and ISO 23977-2:2020 “plastics—determination of the aerobic biodegradation of plastic materials exposed to seawater—Part 2: method by measuring the oxygen demand in closed respirometer”.

The quantified biodegradation property for a formulation may depend on the measurement method and conditions used, the measurement environment and the measurement value related to the degradation process, such as mass loss, DOC, oxygen consumption or carbon dioxide production over time. The measurement method and the measured characteristics may be provided as metadata per measurement point related to biodegradability.

Generally, the formulation can be any formulation. A formulation comprises of at least two components that can refer to any chemical entity. For example, the component can be small molecules, polymers or the like. However, the components can themselves also be more complex chemical products. In a preferred example, the formulation defines a packaging of a product.

In a first step the method comprises providing a target biodegradability that is indicative of a biodegradation characteristic of a formulation. In particular, the providing can refer to receiving the target biodegradability from an input of a user using, for instance, a respective input unit. Moreover, the providing can also refer to accessing a storage unit on which a target biodegradability is already stored. Further, the providing can also comprise receiving a target biodegradability, for instance, via a network connection from other sources and providing the received biodegradability. Generally, the target biodegradability can refer to one target value, for instance, a target half-life of a formulation in a specific habitat, or can refer to a value range that should be met by the formulation in a specific habitat. Moreover, the target biodegradability can also refer to any kind of target function, for instance, a timely sequence of biodegradations. For example, the target biodegradability can indicate that the target formulation shall have a first biodegradability value range during a first time range and then a second target biodegradability value range during a following time range. Moreover, a target degradation function can take the specific interaction of the biodegradabilities of the components of a target formulation into account. For example, a target function can determine that in a first time range first an outer component of a package should biodegrades and in a later second time range an inner component of the package should biodegrade. Such more complex target biodegradabilities can be advantageous in cases in which it is desired that a formulation is not biodegraded in a specific habitat for some time, for instance, for the average usage time of the formulation, and then biodegrades fast in the same or another habitat. Moreover, the target biodegradability can also refer to a target biodegradability of each component of the formulation or a target biodegradation of combinations of the components of the formulation. In particular, in cases in which the components of a formulation fulfil different technical functions in an intended application it can be useful to also determine the specific the biodegradation of each component. For example, for a packaging of a food product, a component in contact with the food product should not biodegrade in the habitat provided by the food product, e.g. a milk environment, but should biodegrade later, for example, in a compost habitat together with the other components of the formulation.

The method comprises providing a digital representation of a potential target preparation specification of a potential target formulation. In particular, the providing can refer to receiving the digital representation from an input of a user using, for instance, a respective input unit. Moreover, the providing can also refer to accessing a storage unit on which the digital representation is already stored. The digital representation of potential target preparation specification can be any representation that provides information that allows to define and prepare the potential target formulation. Moreover, it is preferred that the digital representation comprises and/or allows to derive respective characterizing parameters, for instance, physicochemical characteristics of components of the potential target formulation. Preferably, the digital representation comprises an indication on characterizing parameter referring to the chemical structure of the at least two components of a potential target formulation and a measure for a quantity of the at least two components in the potential target formulation for defining the formulation. A measure for a quantity may be a mass, a volume or the like. Moreover, the digital representation may comprise as characterizing parameter derivatives of the chemical structure of the at least two components such as a quantitative ratio of the at least two components. More preferably, the digital representation may indicate as characterizing parameters components, quantities of components and a morphology of the potential target formulation. A morphology of a formulation may be suitable for describing the mixture of the at least two components. A morphology may be described by morphology descriptors. Further, a morphology may comprise a geometrical measure for describing the result of mixing of the at least two components of the formulation. A result of mixing of the at least two components may be indicated by at least one phase present in the formulation and the relation of at least two phases if at least two phases are present. A relation of at least two phases may indicate the kind of phases present in the formulation and the arrangement of the at least two phases in relation to each other. Further, the digital representation may indicate as characterizing parameters an arrangement of the two component in the formulation. An arrangement may indicate the shape of at least one phase incorporated at least partially into another phase, the size of the incorporated component/structure, e.g. a size of a capsule, the frequency/density of the incorporated component/structure or the like. In an example, a potential target formulation may comprise two phases, a hydrophilic phase with component A and a hydrophobic phase with component B. The phases may be mixed only by a fraction, e.g. under the help of a mixing agent or stirring/shaking the formulation. A part of component A may be incorporated in the phase of component B as spherical drops. Hence the relation of the at least two phases may indicate the size of the drops and the frequency of the drops in the phase with component B. Morphology may be described with a descriptor. Morphology may be described via the at least one phase present in the formulation. The at least one phase present in the formulation may be described by its dimensions, the shape associated with the phase, the aggregation phase, the components present in the phase, a measure for the quantity associated with the components present in the phase or the like.

Additionally or alternatively, the digital representation can indicate as characterizing parameters components, quantities of components and conditions of processing. The conditions of processing may be described by mixing instructions. Mixing instructions may be described by mixing descriptors. Mixing instructions may be indicated by a sequence of adding the at least two components and the conditions of mixing. Conditions of mixing may include details regarding stirring, temperature, pressure, atmosphere or the like. Mixing instructions significantly influence the physicochemical properties of a formulation and thus, the biodegradation. For example, adding component C to component A and B while stirring may be different from adding component B to component A and C while stirring since A and B may establish different intermolecular interactions than A and C. Different intermolecular interactions may lead to different arrangement of the components and thus different formulations.

Additionally or alternatively, the digital representation may further indicate as characterizing parameters storage conditions. Storage conditions may refer to temperature, pressure, atmosphere and time interval associated with storing the formulation.

The morphology or the conditions of processing are advantageous for determining a preparation specification since in an example with a solution comprising capsules the surface of the capsules may be degraded first followed by the inside of the capsules once reached by the microorganisms. Since in such situations the morphology as a result of the conditions of processing is key when determining the part of the formulation to be degraded. Consequently, a target biodegradability may be provided based on the sequence of access to the at least two components in a formulation. The digital representation may thus indicate the sequence of access to the at least two components in a formulation. Also, a relation between the at least two phases may indicate the sequence of access to the at least two components in a formulation.

Additionally or alternatively, the digital representation of a formulation can be indicative of cooperative effects associated with the at least two components as characterizing parameter. Cooperative effects can be synergistic or antagonistic. Cooperative effects become apparent when comparing the biodegradability of the sole components compared to the biodegradability of the mixture of components. In situations with more than two components, cooperative effects even become apparent when comparing biodegradability associated with at least two components selected out of the more than two components with the biodegradability associated with the more than two component formulation. By doing so, a more accurate and realistic description of formulations is achieved.

Preferably, the digital representation comprises as characterizing parameters physicochemical characteristics of the potential target formulation and/or of at least one component of the potential target formulation In particular, the physicochemical characteristics of a formulation can be quantified by physicochemical parameters. Preferably, the digital representation directly comprises the physicochemical parameters, preferably, referring to descriptors. In particular, the physicochemical parameters are indicative of parameters quantifying the physicochemical characteristics of the formulation and/or a component of the formulation. In this context, the term “physicochemical characteristics” refers to physical and/or chemical characteristics of the formulation and/or a component of the formulation. However, the digital representation can also be provided such that it allows to derive the physicochemical characteristics, for example, in form of formulation descriptors, for instance, by providing a representation of a potential target preparation specification for which respective physicochemical characteristics are already stored or can be determined, for instance, by respective descriptor calculations. Preferably, the digital representation refers to at least one of a recipe, a structural formula, a brand name, an IUPAC name, a chemical identifier and a CAS number of the formulation.

The potential target preparation specification can also be regarded as a starting preparation specification that indicates for which formulation or in which region of a potential formulation space the biodegradability should be determined first in the search for a preparation specification that leads to a formulation that comprises the target biodegradability. Generally, the digital representation of the potential target preparation specification can be provided by a user or automatically, for example, in accordance with predetermined rules or also arbitrarily. For example, the user can select a promising potential target preparation specification as starting point. However, also an arbitrary target preparation specification can be utilized or a set of rules can be utilized for providing a potential target preparation specification without user intervention. Preferably, the potential target preparation specification is provided based on rules taking constrains on a potential target preparation specification space, i.e. target formulation space, into account.

Preferably, the physicochemical parameters refer to at least one of constitutional descriptors, count descriptors, list of structural fragments, fingerprints, graph invariants, 3D-descriptors and/or higher dimensional descriptors that are indicative of parameters quantifying physicochemical characteristics of the formulation and/or components of the formulation. In a preferred embodiment the descriptors refer to 3D descriptors, in particular quantum chemical descriptors. Moreover, the inventors have found that in particular a molar mass describes the biodegradation of a formulation and/or a component of the formulation very accurately. Thus, it is in particular preferred that the physicochemical parameters comprise a molar mass of the formulation and/or a component of the formulation. In the following the possible physicochemical parameters are defined in more detail.

A constitutional descriptor can refer to any of a potential, average molecular weight, polydispersity, charge, spin, boiling point, melting point, enthalpy of fusion, dissociation constant, Hansen parameter, protic, polar and dispersive contributions, Abraham parameter, retention index, TPSA, receptor binding constant, Michaelis-Menten constant, Inhibitor constant, Mutagenicity, LD50, bioconcentration, toxicity, biodegradation profile and viscosity. A count descriptor can refer to any of a sum of atomic electro negativities, a sum of atomic polarizabilities, an amount of components, a ratio of amounts of components, a number of atoms and non H-atoms, a number of H, B, C, N, O, P, S, Hal and heavy atoms, a number of H-donor and H-acceptor atoms, a number of bonds, non-H or multiple bonds, a number of double, triple and aromatic bonds, a number of functional groups, a ratio of functional groups, a sum of bond orders, an aromatic ratio, a number of rings or circuits, a number of unpaired electrons, a number of rotatable bonds, rotatable bond fractions, and a number of conformers.

Physicochemical parameters referring to a list of structural fragment descriptors can refer to at least one of a list of molecular fractions, a list of functional groups, a list of bonds, and a list of atoms. Fingerprint descriptors comprise preferably, at least one of MACCS keys, preferably, in bit format or total amount format, Morgan and other circular fingerprints, preferably, in bit format or total amount format, topological torsion, atom pairs, infrared and related spectra, fingerprint count, PubChem fingerprint, substructure fingerprint, and Klekota-Roth fingerprint. Graph invariants/topological indices descriptors comprise preferably at least one of topostructural indices and topochemical indices.

In a preferred embodiment the formulation physicochemical parameters are 3D descriptors comprising at least one of a volume as sum overall atoms, a mean volume per atom, an area as sum overall atoms, an area as mean per atom, an area over all atoms, an area as mean per atom, a solvent accessible surface, a dispersion energy, a dielectric energy, a H-donor, H-acceptor, polar and non-polar surface area, an atom resolved H-donor, H-acceptor, polar and non-polar surface area, a shape, a sphericity, dipole and higher electric moments, polarizability, dielectric energy, protic, polar and non-polar surface area, orbital energies and orbital gaps, ionization energy, electron affinity, hardness, electronegativity, electrophilicity, excitation energies and intensities, infrared and ultraviolet absorption bands, reactivity measurements, redox potential, bond criterial points, partial charges, charge surface areas, atomic orbital contributions, bond orders, atom radius. In particular, it is preferred that the formulation physicochemical parameters refer to 3D descriptors comprising at least one of a sum of a volume over all atoms, a mean of a volume per atom, a sum of the area over all atoms, a mean of an area per atom, a solvent accessible surface, a dispersion energy, a dielectric energy, a H-donor, H-acceptor, polar and/or non-polar surface area, atom resolved H-donor, H-acceptor, polar and/or non-polar surface area, shape, sphericity, cone angles, polarizability, dielectric energy, protic, polar and/or non-polar surface area, excitation energies and intensities, infrared and/or UV absorption bands, reactivity measurements, particle charges and/or charge surface areas. A preferably utilized higher dimensional descriptor can comprise at least one of a conformational partition function, solubility, vapor pressure, activity coefficient, diffusion coefficient, partition coefficient, interfacial activity, rotational constant, moment of inertia, radius of gyration, compositional drift of formulation, density, viscosity, conformer weighted volume and area, conformer weighted H-donor, H-acceptor, protic, polar and/or non-polar surface area, charge distribution, conformational dipole moment and molecular refraction. Preferably higher dimensional descriptors are utilized that comprise at least one of solubilities, vapor pressure and activity coefficients, interfacial activity, conformer weighted H-donor, H-acceptor, protic, polar and non-polar surface area, and charge distribution.

In an embodiment, the physicochemical parameters are determined based on the components of the formulation. For example, the digital representation is indicative of the components of the formulation and the method further comprises classifying the components of the formulation into predetermined component classes, for example, solvents, surfactants, pigments, etc. These classes can be predetermined by a respective expert user or can be learned during the training process of the biodegradation model. The physicochemical parameters can then be determined based on the component classes. In particular, a physicochemical parameter for a component class can be derived based on the values of the physicochemical parameter of the components belonging to the component class. For example a weight % weighted average, a maximum or minimum value, a median, a total amount, etc. can be determined as physicochemical parameter for each component class. If more than one physicochemical parameter is provided for the components in a component class this can be performed for each component. Predetermined rules for a respective class can determine how a respective physicochemical parameter is derived from the physicochemical parameters of the components in the class. The physicochemical parameters determined for each class are then the physicochemical parameters utilized in the biodegradation model.

The method further comprises providing a biodegradation habitat, wherein a biodegradation habitat is indicative of habitat descriptor values of habitat descriptors influencing a biodegradation of a formulation in the respective habitat. In particular, the providing can refer to receiving the biodegradation habitat from an input of a user using, for instance, a respective input unit. Moreover, the providing can also refer to accessing a storage unit on which the biodegradation habitat is already stored. Furthermore, the providing can also refer to a presetting of a biodegradability habitat. For example, if the method is utilized in a very specific context that is only sensible with one specific biodegradation habitat, the respective biodegradation habitat can be preset and thus has not to be provided as specific input. Further, the providing can also comprise receiving directly the habitat descriptor values of the habitat descriptors, for instance, via a network connection, from other sources and providing the received habitat descriptor values of habitat descriptors as biodegradation habitat. The provided biodegradation habitat can refer to a general habitat, for instance, can refer to a marine habitat, wherein respective habitat descriptor values for the habitat descriptors for this habitat are then already stored on a respective storage which can be accessed. However, the provided biodegradation habitat can also directly comprise the respective habitat descriptor values for the biodegradation habitat to provide a further specification of the biodegradation habitat. Moreover, the providing of a biodegradation habitat can include providing a digital representation of the biodegradation habitat, wherein the digital representation can then be indicative of respective habitat descriptor values of habitat descriptors influencing a biodegradation of a formulation in the respective habitat. Further in a preferred embodiment the habitat is derived from the digital representation of the potential target preparation specification. For example, a biodegradation habitat may be indicated by the phases present in the respective potential target formulation and the aggregation phase of the formulation. Moreover, in an embodiment different habitats can be provided or derived for different components of the potential target formulation. For example, if for a specific application it is expected that different components will be subjected to different habitats during the live of the formulation.

Generally, the habitat descriptors are indicative of environmental characteristics of the habitat. In particular, the environmental characteristics of a biodegradation habitat can influence a biological activity in the respective habitat, for example, can influence a presence, grows or absence of specific bacteria. Thus, the environmental characteristics defined by the habitat descriptors indirectly also influence the biodegradation of a formulation in the respective habitat. For example, if a formulation is biodegradable by a specific bacterium that needs a specific salt concentration, the formulation and/or a component of the formulation will biodegrade fast in a habitat providing such a salt concentration, like a marine habitat, but will biodegrade much slower in a habitat with not the right salt concentration, like waste water.

Preferably, the biodegradation habitat refers to any one of a marine habitat, a waste water habitat, a limnic habitat, a compost habitat or a soil habitat. In a preferred embodiment, the biodegradation habitat refers to a marine habitat and wherein the habitat descriptors refer to at least one of a salt concentration, a sedimentation type, oxygen level, location, sample depth, a water temperature, a nutrient concentration, a pH value, an environmental type and a microbial community. In a further preferred embodiment, the biodegradation habitat refers to a limnic habitat and wherein the habitat descriptors refer to at least one of a salt concentration, a sedimentation type, oxygen level, location, sample depth a water temperature, a nutrient concentration, a pH value, an environmental type and a microbial community. In a further preferred embodiment, the biodegradation habitat refers to waste water and the habitat descriptors refer to at least one of a water temperature, a microbial community, a sludge concentration, a nutrient concentration, a pH value, a test duration and an enzyme environment. In a further preferred embodiment, the biodegradation habitat refers to soil and the habitat descriptors refer to at least one of a temperature, a sand content, a pH value, a moisture content, a nutrient concentration, a microbial community and an enzyme environment. In a further preferred embodiment, the biodegradation habitat refers to compost and the habitat descriptors refer to at least one of a temperature, compost activity, a pH value, a moisture content, humidity, compost maturity, compost composition, compost origin, a nutrient concentration, a microbial community and an enzyme environment. Generally, the habitat can also refer to a habitat of a standard test utilized for determining biodegradability of a formulation. For example, standard tests as defined by ISO13432, ISO14852, ISO14855, ISO17556 and OECD 301 also define a specific habitat in which the biodegradation takes place. Thus the providing of the biodegradation habitat can also comprise providing, for instance, selecting via a user input, one of the standard tests, wherein the habitat descriptors then refer to the specific characteristics of the test, i.e. of the test environment and thus test habitat. Moreover, the habitat can also be defined by the biodegradation of a reference formulation or other reference chemical. In this case the habitat can be provided by providing the reference and its biodegradation. In this case the reference and its biodegradation are indicative of the habitat descriptors.

The method further comprises providing a biodegradation model based on the provided biodegradation habitat. In particular, it is preferred that the providing of the biodegradation model refers to a selecting of a biodegradation model based on the provided biodegradation habitat. For example, a plurality of biodegradation models can be stored on a biodegradation storage, wherein each biodegradation model has been trained for one or more different biodegradation habitats. Preferably, each biodegradation model is, in particular, trained for different values or value ranges of habitat descriptor values of a biodegradation habitat. Based on the provided biodegradation habitat indicative of the habitat descriptor values, a respective suitable biodegradation model can then be selected from the plurality of biodegradation models. For example, a biodegradation model is suitable if the indicated habitat descriptor values fall within the ranges of the habitat descriptor values for which the biodegradation model has been trained. For example, a respective lookup table can be provided that allows for an easy comparison between the indicated habitat descriptor values and the descriptor value ranges for which the biodegradation models stored on the storage have been trained such that directly a suitable biodegradation model can be selected. However, in another embodiment the providing of a biodegradation model based on the provided biodegradation habitat can also refer to a user selection of the biodegradation model. For instance, the user can be provided with a preselection of biodegradation models that refer to the provided biodegradation habitat and then be allowed to select the respective biodegradation model that should be utilized. Generally, the possible stored biodegradation models refer to biodegradation models that have already been parameterized based on a respective training data set for one or more habitats. Since the training data sets utilized for parameterizing a biodegradation model are historical data, as described in more detail below, the biodegradation models can be trained and thus generated at any time before the determination of a specific biodegradation for a specific formulation, and after the training be stored on a respective database. However, the training and thus the generation of a biodegradation model can of course also be performed at the time that it is determined that a specific biodegradation model, for instance, for a specific habitat, is needed.

In an embodiment, the biodegradation model is parameterized based on a training data set comprising a measured biodegradations in a respective habitat associated with respective formulations in the training data set. The measured biodegradation may be measured with respect to a respective habitat utilizing a predetermined the biodegradation test method, for instance, any of the test methods described above. The biodegradation model therefore represents the measured biodegradability of the training formulations.

The provided biodegradation model is then adapted to determine a biodegradability of a formulation in the respective biodegradation habitat. In particular, the biodegradation model is a data driven model that is parameterized with respect to the biodegradation habitat such that it can determine the biodegradability of a formulation based on the by the digital representation. Preferably, the biodegradation model is trained to determine the biodegradation based on the characterizing parameters of the formulation, preferably, based on components of the formulation and the quantities of the components derivable from the digital representation. Additionally, also at least one of a morphology, processing condition and storage condition derivable from the digital representation can be utilized as characterizing parameters and as input for the biodegradation model to determine the biodegradation. With respect to the components of the formulation, the components themselves can be used as input to the biodegradation model. However, also parameters derivable for the components can be utilized as input characterizing parameters, i.e. as characterizing parameter being input to the biodegradation model. For example, at least one of a chemical structure and physicochemical parameters of the component can be utilized as input characterizing parameters. Moreover, the quantities of the components provided as input characterizing parameters can refer to any of a mass, volume and ratio of the respective components. For the morphology the input characterizing parameters can refer to morphology descriptors, for instance, as already described above. The processing conditions can refer to at least one of mixing conditions. The storing condition can refer to at least one of a temperature, pressure, atmosphere and time interval associated with storing the formulation. The term “such that” is to be interpreted here that the parameterization adapts and thus enables the biodegradation model to provide the biodegradability with respect to a habitat when provided with formulation physicochemical parameters as input. For example, the biodegradation model relates formulation physicochemical parameters of historic digital representations of preparation specification and historic digital representations of habitats to a biodegradability. This allows that, based on a target biodegradability, a digital representation of the preparation specification may be determined. The term “data driven” is used here to emphasize that the model is mainly based on respective data input and not, for instance, on intuition, personal experience or knowledge. Preferably, the biodegradation model refers to a machine learning based model that is based on known machine learning algorithms, like neural networks, regression models, classification algorithms, etc. It has been found that for most applications in this context, in particular, regression models based on Linear Regression, Random Forests, Boosted Trees, Lasso, Ridge Regression and MARS algorithms are suitable, whereas for classification models, in particular, Random Forests, Logistic regression and SVM algorithms are suitable. Generally, the biodegradation model is parameterized during a training process in which digital representation of formulations or one or more characterizing parameters derived from the digital representation, as described above, are utilized together with corresponding biodegradabilities for specific biodegradation habitats. Based on such a training data set that is specific for a biodegradation habitat, for instance, for specific habitat descriptor value ranges and/or values, the respective parameters of the data driven model can be determined utilizing known training methods such that the biodegradation model is also able to determine a biodegradation of formulations that are not part of the training data set.

Moreover, in a preferred embodiment, the biodegradation model can also be adapted to determine the biodegradation for a formulation further based on habitat descriptor values as input. In particular, the biodegradation model can be trained by utilizing a training data set comprising formulation and/or derivable characterizing parameters as described above and associated biodegradabilities for a specific habitat, as described above, leading to a biodegradation model that indirectly takes the specific habitat into account. However, the training data set can optionally also comprise specific habitat descriptor values of a respective habitat. In this case, the biodegradation model can be trained such that in addition to the formulation and/or derivable characterizing parameters as described above also habitat descriptor values can be provided as input, wherein the biodegradation model then determines the biodegradability further based on the habitat descriptor values. This has the advantage that the biodegradability can be determined even more accurately, in particular, in cases in which the biodegradation strongly depends on the specific habitat descriptor values of the habitat. For example, in a marine habitat a temperature or salt concentration can strongly deviate for different regions of the world, wherein for some formulations this can also lead to different biodegradabilities. Thus, for such cases it can be advantageous to directly provide the habitat descriptor values as input to the biodegradation model. However, it is also possible instead of providing the habitat descriptor values as input to biodegradation model, to train two different biodegradation models and indirectly treat the different regions as different habitats.

Further, the method comprises determining the biodegradability of the potential target formulation based on the provided biodegradation model and the digital representation. In particular, as described above the digital representation of the potential target preparation specification, i.e. the provided components and quantities of the components of the formulation, can be provided as input characterizing parameters to the biodegradation model. However, also further characterizing parameters can be provided by or derived from the digital representation of the potential target preparation specification and utilized as input, as described above. The biodegradation model then provides the biodegradability of the potential target formulation as output. If the digital representation does not directly comprise characterizing parameters, the determining of the biodegradability can comprise also determining firstly the characterizing parameters, for instance, as described above. Such determined characterizing parameters can then be provided to the biodegradation model as input.

The determination of the biodegradability utilizing the biodegradation model can be regarded as a virtual measurement of the biodegradability. In particular, the biodegradation model is based on measurement data, for example, measured biodegradabilities of formulations utilized for the training of the biodegradation model. Thus, the biodegradation model comprises the information provided by these previous measurements. Moreover, the physicochemical parameters can in some cases also refer to measured characteristics of the formulation. Accordingly, also the determined biodegradability of new formulation determined utilizing the biodegradation model can be regarded as being based at least partly on measurement results.

In a following step, the determined biodegradability of the potential target formulation is compared with the target biodegradability. Based on the comparison it is decided if the potential target formulation is determined as the target formulation and the potential target preparation specification is determined as the target preparation specification, wherein in this case the iteration can stop at this point. Moreover, based on the comparison it can also be determined to provide a new potential target preparation specification of a new potential target formulation and to repeat the determination of the biodegradability utilizing the new potential target preparation specification of the new potential target formulation. Thus, at this point an iteration is performed in which the determination of the biodegradability using the biodegradation model and the characterizing parameters of potential target formulations is repeated until one of the potential target formulations is determined as the target formulation. In particular, the comparison can comprise determining whether the determined biodegradability of a potential target formulation lies within a predetermined range around the target biodegradability, wherein in this case the target can be regarded as being fulfilled and the potential target formulation is determined as target formulation. If the determined biodegradability lies outside of the predetermined range around the target biodegradability, it is determined that the target is not fulfilled and a new potential target preparation specification of a new potential target formulation is provided that might fulfil the target biodegradability.

Generally, the performed iteration can refer to an arbitrary search of the potential target formulation space or to a directed search. For example, a new potential target preparation specification or a new potential target formulation can simply be selected arbitrarily from a huge amount of in-silico generated potential target formulations. However, also specific rules for generating a new potential target formulation and thus a new potential target preparation specification can be applied based on the comparison between the determined biodegradability and the potential target formulation, with or without considering the simultaneous optimization of additional target properties of the formulation. For example, the components can be amended or changed. Moreover, not only the components can be optimized, but also the interplay between the components that can be essential for the formulations. Components of formulations are usually widely known materials whose new combinations provide the effects the formulation is known for. Thus, optimization of the sole components in many cases cannot provide the changes needed for meeting the target biodegradability. Thus, instead of designing a new component for a formulation with the means disclosed herein instead the preparation specification can be changed to arrive at the target biodegradability. By doing so, resources regarding preparation of new materials are saved and standard chemicals can be combined more efficiently. Generally, known methods for generating new potential target formulations and/or new potential target preparation formulation can be utilized, for example, evolutional algorithms or Bayesian optimizers can be used.

The iteration can then be performed over the steps of determining the biodegradability of the new potential target formulation by utilizing the biodegradation habitat and the characterizing parameters of the new potential target formulation as descript above. Optionally, also a determination of characterizing parameters from the digital description of the new potential target preparation specification can be part of the iteration, if the characterizing parameters are not already provided with the digital description of the new potential target preparation specification. Moreover, it is preferred that the same biodegradation model is used in all iteration steps for determining the biodegradability. However, in some cases also different biodegradation models can be used in different iteration steps. For example, if other characterizing parameters for the new potential target formulation are utilized also another biodegradation model can be more suitable.

After the iteration has stopped, for instance, after the potential target formulation has been determined as the target formulation, or if no new potential target formulation can be selected or generated, the result of the iteration can be provided to a user. For example, if none of the possible potential target formulations has met the target biodegradability, the user can be notified of the failure of determining a target formulation. In case a target formulation can be determined, the target formulation can be provided to the user as output. For example, the determined target formulation and target preparation specification can then be provided to an output unit or to a computing unit for further processing. Preferably, the providing of the target preparation specification and the target formulation leads to a further processing utilizing the target preparation specification.

Preferably, the processing of the target preparation specification comprises determining control signals for controlling a production process based on the determined target preparation specification. Preferably, the production process refers to a production process of the target formulation utilizing the target preparation specification. Moreover, it is preferred that the target preparation specification refers to a machine executable preparation specification of the target formulation such that the control signals can directly refer to a controlling of respective laboratory or process equipment allowing to execute the preparation specification to produce the formulation. In an embodiment, the providing of the target preparation specification of the target formulation comprises providing control signals adapted for controlling an industrial plant for producing the target formulation in accordance with the target preparation specification.

In a preferred embodiment, the digital representation of the potential target preparation specification is indicative of the components of the potential target formulation and the biodegradability is determined for each of the components individually, wherein an overall biodegradation of the formulation is determined based on the determined biodegradabilities of the components and the overall biodegradability is then compared to the target biodegradability. Preferably, the overall biodegradability is set to the biodegradability of the component formulations with the lowest biodegradability. However, the overall biodegradability can also be determined based on other predetermined rules, for example, as an average value of all component biodegradabilities, Moreover, the digital representation can further be indicative of the quantities of the components in the formulation. In this case the overall biodegradability can further be determined based on the quantities, for example, as a weighted average, wherein the weights are determined by the quantities. Moreover, in an embodiment the biodegradability of each component can also be compared to the target biodegradability individually, and respective rules can be utilized to determine under which conditions the target biodegradability is fulfilled. For example, the rules can determine that the biodegradabilities of all components have to meet the target biodegradability or that only some components have to meet the biodegradability. In particular, a target biodegradability can be provided specifically for at least some of the components such that the formulation meets the target biodegradability if the specific biodegradabilities are met by the respective components. Generally, for these embodiments, the biodegradation of each component can be determined as already described above. For example, the same biodegradation model can be utilized for each component and respective characterizing parameters of the components can be provided as input to the respective biodegradation model. However, for different components also different biodegradation models can be used, for instance, biodegradation models specifically trained for a respective component.

In an embodiment, further a target application of the formulation is provided referring to an intended application of the target formulation, wherein the biodegradation habitat is provided based of the target application. A target application of a formulation can refer, for instance, to an intended application context of the formulation, for example, if it is intended to utilize the formulation as a coating, in personal care products, in a washing detergent, in lubricant or in a packaging of a product. Such target applications indicate specific biodegradation habitats. For example, for a packaging of a product it could be interesting if a formulation biodegrades in a compost. In another example, if the target application refers to utilizing the formulation in personal care products, it is very likely that the formulation will sooner or later be found in a water environment. Thus, a respective target application is indicative for a respective biodegradation habitat. In this context, a predetermined list can be provided on a storage on which respective target applications and corresponding biodegradation habitats are stored. A target application for a formulation can then be provided, for instance, by providing the list of target applications to a user and allowing the user to select a respective target application, wherein a respective target application is connected to one or more biodegradation habitats. A target formulation can then be determined for each of the biodegradation habitats to which the target application is connected or again a user can select a respective biodegradation habitat connected with the target application. Additionally or alternatively, information indicative of an intended end-of-life treatment of the formulation can be provided. For example, an end-of-life treatment can be indicative of, whether the formulation is intended to biodegrade in a specific environment, or should be subjected to a specific treatment, for example, in a bioreactor. Thus, also the information of the intended end-of life treatment can be utilized to determine a biodegradation habitat for the formulation, as described above.

In an embodiment, further information indicative of an accessible surface area of the formulation in its intended form is provided, wherein the biodegradation model is further trained to determine a biodegradability based on the accessible surface area, and wherein the method further comprises determining the biodegradability further on the accessible surface area. For example, the information can refer to whether the intended product is provided in a solid, pulverized, foamy, pelletized, or any other form. Preferably, the information is indicative of a surface area of the product per mass or a geometry of a smallest independent part of the product. Generally, although the biodegradability of a formulation is an intrinsic characteristic of the formulation, the exact timing of the biodegradability of a product comprising the formulation can also depend on the surface area that can be accessed, for instance, by microbial components of the habitat responsible for the biodegradation. Thus, further determining the biodegradability based on a surface area of a product comprising the formulation allows to increase the accuracy in the prediction of the biodegradability of the final product and thus also to increase the accuracy of determining a suitable target formulation for the final product.

In an embodiment, a target technical application property for the target formulation is provided and the potential target preparation specification is provided based on the provided target technical application property such that the potential target formulation fulfils the provided target technical application property. In particular, the technical application property can refer to any property of a formulation and/or a substance consisting at least partly of the formulation that allows to assess a technical applicability of the respective formulation as provided after its preparation. Preferably, the technical application property comprises at least one of mechanical properties, optical properties, physicochemical properties, chemical properties and biological properties. Generally, mechanical properties can refer to any of adhesion, tensile strength, stiffness, hardness, shrinkage, elongation, split tear, tear-strength, rebound, compressibility, abrasion, spillage, morphology, haptic properties, stress at break, elongation at break, granulometry and a degree of filling. An optical property can generally comprise any of coloration, turbidity, opaqueness, lucidity, reflection, appearance, absorption, scattering, color strength, cloud point, matting degree, optical density, spectra, refractive index. Moreover, a physicochemical property can refer to any of density, viscosity, K-value, molar weight, dispersity, molar mass distribution, particle size distribution, solubility, partition coefficients, interfacial properties, surface tension, dispersibility, storage stability, odor, segregation, coagulation, electric conductivity, electric capacity, surface area, flow time, vapor pressure, VOC, solid content, hygroscopicity, magnetism, miscibility, thixotropy, phase transition properties, glass transition temperature, corrosion inhibition, solvent separation, aggregation, self-heating ability, impact sensitivity, loss on drying, angle of response, electrostatic charge, minimum film-forming temperature, and charge density. The chemical property can comprise any of functional group count, atom type count, functional group density, atom type density, chemical resistance, reaction timing, demolding time, growing, hard/soft segment content, crystallinity, reaction temperature, reaction pressure, decomposition, thermal decomposition, photodegradation, acidity, pKa, pH, moisture/water content, flammability, burning rate, selfignition, flash point, formation of flammable gases, reaction to fire, deflagration rate, residual monomer count, side product formation, salt content, temperature tolerance, oxidizing properties, reduction properties, reactivity, ash content, nonvolatile matter content, stability, chelating ability, calorific value, saponification value. Further, the biological property can comprise any of biodegradability, biological resistance, toxicity, biotransformation, ecotoxicology, sensitization, bacterial count, enzyme activity, distribution in environment, bioaccumulation, biological exposure. In a preferred embodiment, the technical application property can further refer to a biodegradability, for instance, to a biodegradability in another habitat. For example, the first target biodegradability can then refer to a marine habitat, wherein the second target biodegradability, i.e. in this case the technical application property, can refer to waste water.

The potential target preparation specification is then provided such that the associated potential target formulation fulfils the provided target technical application property. For example, a database can be utilized on which formulations and corresponding technical application properties are already stored and from the database target formulations and associated preparation specifications can be selected that fulfil the provided target technical application property. Generally, the formulations fulfilling the target technical application property can be regarded as forming the potential target formulation space that can be explored during the iteration process for finding the target formulation. From the selected target formulations fulfilling the target technical application property the first potential target formulation and thus the first potential target preparation specification can then be selected.

In an embodiment, the providing of a new potential target preparation specification is based on amending the provided target application property and providing the new potential target preparation specification such that the potential target formulation fulfils the amended target application property. In particular, if the new potential target preparation specification has to be provided, the comparison of the determined biodegradability and the target biodegradability indicates that the determined biodegradability of the current potential target formulation does not fulfil the target biodegradability. In such a case, the new potential target preparation specification and thus a new potential target formulation can be provided such that the new potential target formulation still fulfils the target technical application property, if such a respective formulation exists. However, in many cases it will not be possible to provide such a new potential target formulation or it might not be technically sensible to provide such a new potential target formulation that still fulfils the target technical application property. In these cases it is advantageous to amend the target application property, for instance, to utilize a less strict target technical application property, like amending the target technical application property such that it now refers instead of one specific value to a value range or if it refers to a value range to a wider value range. The new potential target formulation can then be selected or generated such that it fulfils the amended target application property.

In an embodiment, the providing of the potential target preparation specification based on the provided target technical application property comprises utilizing a determination model adapted to determine a technical application property of a formulation based on the digital representation of the formulation, wherein the determination model is a data driven model parameterized such that it determines based on the digital representation comprising the characterizing parameters of the formulation the technical application property associated with the formulation. The determination model can refer to any known data driven determination model that allows to determine the technical application property based on a digital representation of a formulation comprising characterizing parameters. Generally, it is preferred that the determination model follows the same principles as described above with respect to the biodegradability model. In fact, the determination model can be based on or utilize the same machine learning algorithms and training methods, only utilizing different training data, i.e. training data comprising instead of the biodegradability another respective technical application property of a formulation. Thus, all embodiments described above with respect to the biodegradation model can also be realized with respect to the determination model for determining the technical application property. Utilizing such a determination model has the advantage that an iteration can be performed not only over the biodegradability of a formulation but also over one or more further technical application properties in a fast and computationally inexpensive manner leading to a target formulation that not only fulfils a target biodegradability but also the one or more further target technical application properties.

In an embodiment, habitat descriptor values for the habitat descriptors are stored associated with respective geolocations, wherein the providing of a biodegradation habitat refers to providing a geolocation of the habitat and retrieving the habitat descriptor values for the geolocation from storage. Geolocations can refer, for instance, to coordinates, or other regional identifications. For example, a geolocation can refer to the name of a city, country, country region, sea region, geographical feature, etc. Based on such geolocations, respective habitats and/or habitat descriptors, for instance, average values, or minimal and maximal values of the habitat descriptors, can be stored. Thus, by providing the geolocation, the respective habitat descriptor values for this geolocation can be provided. This has the advantage that an exact habitat or exact habitat descriptor values for a region do not have to be known to a user. Thus, the user can simply provide a location for which it is expected that the target formulation might biodegrade in this region.

In an embodiment, the characterizing parameters indicated by the digital representation of the formulation refer at least to one of recipe parameters from the preparation, constitutional descriptors, count descriptors, list of structural fragments, fingerprints, graph invariance, 3D-descriptors and/or higher dimensional descriptors that are indicative of a chemical nature of the formulation and/or component of the formulation. Respective connections of the digital representation with characterizing parameters, for instance, calculated previously, or further information on the formulation, can be stored already and connected with the respective digital representation. For example, if the digital representation refers to a brand name, components, respective structural formulas and quantities, and/or physicochemical parameters or corresponding to the brand name can be stored already, for example, on a storage of the brand name owner.

In a further aspect, an interface method for providing an interface is presented, wherein the interface method comprises a) receiving as input a target biodegradability, digital representation and a habitat via a user interface and providing the received target biodegradability, digital representation and the habitat to a processor performing the method as described above, and b) providing the target preparation specification of the formulation as result, wherein the result is received from the processor performing the method as described above.

In a further aspect, a computer implemented training method for training a data driven based biodegradation model for parameterizing the biodegradation model is presented, wherein the training method comprises a) providing training data associated with a predetermined biodegradation habitat, wherein the training data comprises i) digital representations of a plurality of training formulations, and ii) a biodegradability for the respective biodegradation habitat associated with each training formulation, b) providing a data driven based trainable biodegradation model, c) training the provided data driven based biodegradation model based on the provided training data such that the trained biodegradation model is adapted to determine a biodegradation of a formulation based on the digital representation of the formulation, and d) providing the trained biodegradation model.

In a further aspect, an apparatus for determining a target preparation specification indicative of a target formulation comprising a target biodegradability is presented, wherein the apparatus comprises a) a target biodegradability providing unit for providing a target biodegradability, wherein a biodegradability is indicative of a biodegradation characteristic of a formulation, b) a digital representation providing unit for providing a digital representation of a potential target preparation specification of a potential target formulation, c) a habitat providing unit for providing a biodegradation habitat, wherein the biodegradation habitat is indicative of habitat descriptor values of habitat descriptors influencing a biodegradation of a formulation in the respective habitat, wherein the habitat descriptors are indicative of environmental characteristics of the habitat, d) a model providing unit for providing a biodegradation model based on the provided biodegradation habitat, wherein the biodegradation model is adapted to determine the biodegradability of a formulation in the respective biodegradation habitat, wherein the biodegradation model is a data driven model parameterized with respect to the biodegradation habitat such that it determines a biodegradability of a formulation based on the the digital representation, e) a biodegradability determination unit for determining the biodegradability of the potential target formulation based on the selected biodegradation model and the digital representation, and f) a iteration control unit for comparing the determined biodegradability of the potential target formulation with the target biodegradability and, based on the comparison, either i) determining the potential target formulation as the target formulation and the potential target preparation specification as the target preparation specification, or ii) providing a new potential target preparation specification of a potential target formulation and repeating the determination of the biodegradability utilizing the new potential target preparation specification of the potential target formulation.

In a further aspect, an interface apparatus for providing an interface is presented, wherein the interface apparatus comprises a) an input interface unit for receiving as input a target biodegradability, a start digital representation and a habitat via a user interface and for providing the received target biodegradability, start digital representation and the habitat to an apparatus as described above, and b) an result interface for providing the habitat descriptor values of the formulation as result, wherein the result is received from the apparatus as described above.

In a further aspect, a training apparatus for training a data driven based biodegradation model for parameterizing the biodegradation model is presented, wherein the training apparatus comprises a) a training data providing unit for providing training data associated with a predetermined biodegradation habitat, wherein the training data comprises i) digital representations of a plurality of training formulations, and ii) a biodegradability for the respective biodegradation habitat associated with each training formulation, b) a trainable model providing unit for providing a data driven based trainable biodegradation model, c) a training unit for training the provided data driven based biodegradation model based on the provided training data such that the trained biodegradation model is adapted to determine a biodegradation of a formulation based on the digital representation, and d) a trained model providing unit for providing the trained biodegradation model.

In a further aspect of the invention a use of the method as described above is presented, wherein the method is used for determining a target formulation comprising a target biodegradability for any of the following i) formulations comprising polyesters, in particular, used for mulch film and packaging applications, e.g. aromatic aliphatic copolyesters, ii) formulations comprising polyalkoxylates, in particular, used for home and personal care applications, iii) formulations comprising polyurethane dispersions, iv) formulations used for aroma applications, v) formulations used for paper coatings for packaging applications based on multilayer blends, and vi) formulations comprising polyurethane used for adhesives.

In a further aspect of the present invention, a system is presented, wherein the system comprises i) a control signal comprising a preparation specification of a formulation indicating one or more ingredients for producing the formulation, wherein the control signals are generated according to the above described method, and ii) the one or more ingredients indicated by the preparation specification in the control signal.

In a further aspect of the invention, a use of a control signal generated according to the above described method for controlling a production process, in particular, a production process comprising the production of a formulation is presented.

In a further aspect of the invention, a control signal is presented, wherein the control signal is generated according to the above described method. Preferably, the control signal comprises a machine executable preparation specification for producing a target formulation.

In a further aspect, a computer program product for determining a target formulation comprising a target biodegradability is presented, wherein the computer program product comprises program code means for causing the apparatus as described above to execute the method as described above.

In a further aspect, a computer program product for training a biodegradation model is presented, wherein the computer program product comprises program code means for causing the apparatus as described above to execute the method as described above.

It shall be understood that the methods as described above, the apparatuses as described above and the computer program products as described above have similar and/or identical preferred embodiments, in particular, as defined in the dependent claims. Moreover, also the training method as described above, the training apparatus as described above, and the training computer program product as described above have similar and/or preferred embodiments, in particular, as defined in the dependent claims.

It shall be understood that a preferred embodiment of the present invention can also be any combination of the dependent claims or above embodiments with the respective independent claim.

These and other aspects of the present invention will be apparent from and elucidated with reference to the embodiments described hereinafter.

### BRIEF DESCRIPTION OF THE DRAWINGS

In the following drawings:

FIG. 1 shows schematically and exemplarily an embodiment of a system comprising an apparatus for determining a target preparation specification indicative of a target formulation comprising a target biodegradability,

FIG. 2 shows schematically and exemplarily a flow chart of a method for determining a target preparation specification indicative of a target formulation comprising a target biodegradability,

FIG. 3 shows schematically and exemplarily a flow chart of a method for training a biodegradation model for determining a biodegradability of a formulation,

FIGS. 4 and 5 show schematically and exemplarily a flow chart of preferred more detailed embodiments of a method for determining a target preparation specification indicative of a target formulation comprising a target biodegradability, and

FIGS. 6 to 8 show schematically and exemplarily a block diagram of a system architecture of a system and apparatus for determining a target preparation specification indicative of a target formulation comprising a target biodegradability.

### DETAILED DESCRIPTION OF EMBODIMENTS

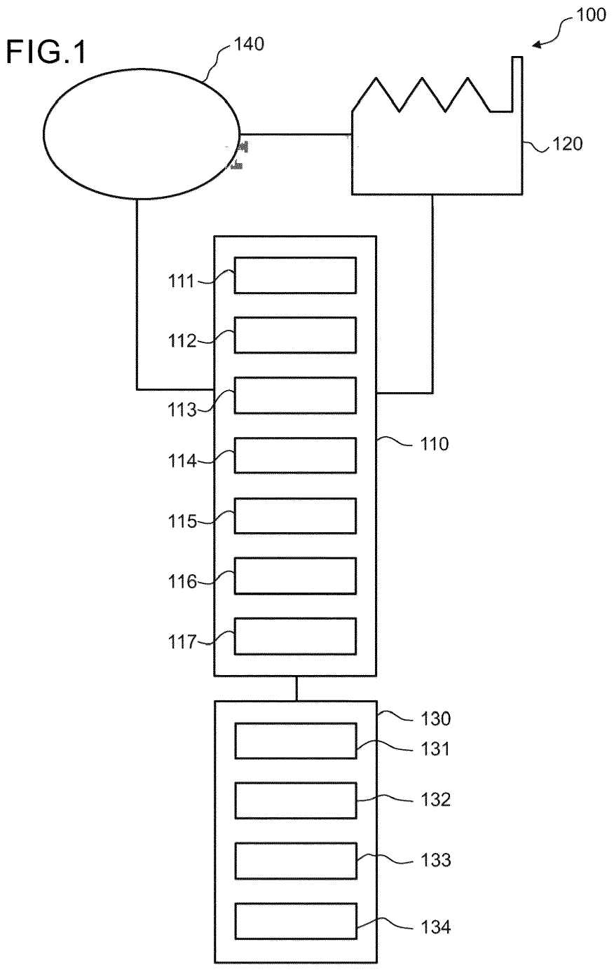

FIG. 1 shows schematically and exemplarily an embodiment of a system 100 comprising an apparatus 110 for determining a target preparation specification indicative of a target formulation comprising a target biodegradability. Further, the system 100 comprises a training apparatus 130 for training a biodegradation model utilized in the apparatus 110, a database 140 on which results of the determining of the target preparation specification can be stored and a production system 120 for producing a product, in particular, comprising the determined target formulation that can be controlled utilizing the determined target preparation specification.

The apparatus 110 comprises a target biodegradability providing unit 111, a digital representation providing unit 112, a habitat providing unit 113, a model providing unit 114, a biodegradability determination unit 115, an iteration control unit 116 and optionally an output and/or control unit 117 that can be adapted to output the determined target preparation specification and/or to provide control signals for controlling a production process of the production system 120 based on the determined preparation specification.

The target biodegradability providing unit 111 is adapted to provide a target biodegradability indicative of desired biodegradation characteristics of a formulation. The target biodegradability providing unit 111 can refer, for instance, to an input unit into which a user can input a respective target biodegradability. Moreover, the target biodegradability providing unit 111 can refer to or can be part of a user interface that allows the user to interact with the apparatus 110 for providing the target biodegradability. However, the target biodegradability providing unit 111 can also refer to or be communicatively coupled with a storage unit on which a target biodegradability, for instance, for a specific application, is already stored.

The digital representation providing unit 112 is adapted to provide a digital representation indicative of a potential target preparation specification of a potential target formulation. The digital representation providing unit 112 can refer, for instance, to an input unit into which a user can input the respective digital representation. Moreover, the digital representation providing unit 112 can refer to or be part of a user interface that allows the user to interact with the apparatus 110 and/or the database 140. However, the digital representation providing unit 112 can also refer to or be communicatively coupled with a storage unit on which the digital representation of the formulation is already stored. Generally, the digital representation can directly comprising characterizing parameters of the formulation, for example, component and quantities of components and/or physicochemical parameters of the respective formulation and/or components of the formulation. However, instead of directly providing the characterizing parameters also only the preparation specification of a formulation can be provided. In this case, it is preferred that the digital representation providing unit 112 is further adapted to determine the characterizing parameters from the preparation specification. In particular, the digital representation providing unit 112 can be adapted to determine characterizing parameters, for instance, by accessing a database on which for a plurality of the most relevant characterizing parameters of a formulation are already stored. The digital representation providing unit 112 is then adapted to provide the digital representation comprising the characterizing parameters, for instance, to the biodegradability determination unit 115.

The habitat providing unit 113 is adapted to provide the biodegradation habitat. The habitat providing unit 113 can refer, for instance, to an input unit into which a user can input a respective biodegradation habitat. For example, a user interface can be provided that allows a user to select from a number of predetermined biodegradation habitats. In a preferred embodiment, the habitat providing unit 113 can be communicatively coupled to or refer to a user interface that allows to indicate a geolocation, for instance, by marking a location on a map, by indicating coordinates, or providing a name of a region, for instance, a political or geological region, wherein the habitat providing unit can then be adapted to provide a biodegradation habitat based on the geolocation. For example, if the geolocation indicates a specific sea region like the Northern Sea or the Atlantic, the habitat providing unit can be adapted to determine as biodegradation habitat a marine habitat.

Generally, a biodegradation habitat is indicative of habitat descriptor values of habitat descriptors influencing a biodegradation of a formulation in the respective habitat. In particular, habitat descriptors are indicative of environmental characteristics of the habitat, for example, for a marine habitat a salt concentration can strongly influence the biodegradation of a formulation in the marine habitat. Specific habitat descriptor values typical for a respective habitat can be stored on a database. However, a user can also input respective specific habitat descriptor values, for example, if it is known that the habitat descriptor values for the respective habitat deviate from the typical habitat descriptor values.

The model providing unit 114 is adapted to provide a biodegradation model based on the provided biodegradation habitat. In particular, it is preferred that the model providing unit 114 is adapted to select the biodegradation model from a plurality of biodegradation models stored already on a database. For example, a biodegradation model can be trained with respect to training data corresponding to one or more specific biodegradation habitats. These specific biodegradation habitats can be defined with respect to specific habitat descriptor values or value ranges that define for which biodegradation habitat the respective biodegradation model is suitable. For example, a lookup table can be provided that allows the model providing unit to select based on the biodegradation habitat, for instance, based on the habitat descriptor values of the biodegradation habitat, which of the biodegradation models is suitable. However, the model providing unit 114 can also comprise or refer to an input unit to which the biodegradation model can be provided, for instance, by a user selection or user input that indicates which biodegradation model should be used.

The biodegradation model is a data-driven model parameterized such that it can determine the biodegradability of the formulation based on the digital representation, in particular, based on the characterizing parameters of the formulation, a biodegradability. Optionally, the biodegradation model can also be trained to further utilize the provided habitat descriptor values as input. In a preferred embodiment, the data-driven model refers to a machine learning model, for instance, utilizing regression model based algorithms or classifier model based algorithms. A regression model based algorithm can be based on any of a neural network algorithm, a Linear Regression algorithm, a LASSO algorithm, a Ridge Regression algorithm, a MARS algorithm, a Random Forest algorithm, and a Boosted Trees algorithm. A classifier based model algorithm can be based on any of a Random Forest algorithm, a Logistic Regression algorithm and a SVM algorithm. The inventors have found that for most applications, in particular, Linear Regression, Random Forest and MARS based algorithms are suitable.

The biodegradation model can be trained, for instance, utilizing training apparatus 130. In particular, the training apparatus 130 comprises a training data providing unit 131 for providing training data for training the data-driven based biodegradation model. The training data comprises a) digital representations of a plurality of training formulations, and b) biodegradabilities associated with each training formulation for one or more different habitats. Optionally, the training data set can further comprise habitat descriptor values of the specific habitat for which the respective biodegradability of a formulation has been determined. Preferably, in the training data the biodegradability provided for each training formulation refers to a biodegradability that is measured in accordance with the same measurement method. However, biodegradabilities can also be provided for different measurement methods, wherein in this case it is preferably clearly indicated which biodegradabilities are associated with which measurement methods, such that the biodegradability model can be trained to differentiate between different measurement methods. Generally, the training data can be designed to cover a predetermined habitat space of a to be trained biodegradation model, wherein the habitat space is defined by the value ranges of the respective habitat descriptors for which the biodegradation model shall be trained. For example, the training data can be designed to cover predetermined formulation types for a pre-determined habitat. Known methods for designing and optimizing training data for a predetermined habitat space can be utilized such that the habitat space is well covered with training data and that random outliers are avoided.

Further, the training apparatus 130 comprises a model providing unit 132 adapted to provide a data-driven based trainable biodegradation model, for instance, a biodegradation model comprising parameters that can be set during the training process for training the biodegradation model. For example, a trainable biodegradation model can already be stored on a storage unit to which the model providing unit 132 can have access for providing the same. Moreover, the training apparatus 130 comprises a training unit 133 for training the provided data-driven based biodegradation model based on the provided training data. In particular, the training can refer to varying the parameters of the biodegradation model based on the respective training data until the biodegradation model is adapted to determine a biodegradability of a formulation based on a digital representation, in particular, based on characterizing parameters. Generally, any know training algorithms for training data-driven, in particular, machine learning based models can be utilized. Preferably, during the training of the biodegradation model also the characterizing parameters of the formulation that have the most influence on the biodegradability in the respective habitat are determined and the model is then trained based on these most influential characterizing parameters. For determining these most influential characterizing parameters, for example, cluster analysis or PCA analysis tools can be utilized. In particular, the characterizing parameters can be utilized to determine the application space of the training data, wherein the application space is then defined by the characterizing parameters of the formulation and the habitat descriptors that are covered by the data. The determination of the most influential characterizing parameters and/or habitat descriptors can then be performed as a dimension reduction of the application space. Then algorithms for optimizing the training data in the application space can be applied, for instance, to cover the application space with as few training data as possible.

The training apparatus 130 then comprises a trained model providing unit 134 that is adapted to provide the trained biodegradation model, for instance, to a storage unit on which respectively trained biodegradation models for different habitat and/or different types of formulations, and/or characterizing parameters are stored. However, the trained model providing unit 134 can also be adapted to directly provide the trained biodegradation model, for instance, to the biodegradation model providing unit 114 of apparatus 110.

In all cases, the biodegradation model providing unit 114 is then adapted to provide a suitable trained biodegradation model to the biodegradability determination unit 115. The biodegradability determination unit 115 can then utilize the biodegradation model and the provided digital representation for determining the biodegradability. In particular, the biodegradability determination unit 115 can be adapted to utilize the characterizing parameters indicated by the digital representation as input to the biodegradation model that has, as already described above, been trained to then provide as output a determination for the biodegradability for which it has been trained.

Further, the apparatus comprises the iteration control unit 116 that is adapted to control an iteration process for determining the target preparation specification. In particular, the iteration control unit 116 is adapted to compare the determined biodegradability of the potential target formulation with the target biodegradability. Based on this comparison, the iteration control unit 116 is then adapted to decide whether a further iteration step is necessary for determining a target preparation specification or if the iteration has reached an end, in particular, if the potential target formulation can be set as the target formulation and thus the potential target preparation specification as the target preparation specification. Preferably, the comparing of the determined biodegradability of the potential target formulation and the target biodegradability refers to determining whether the determined biodegradability lies within a predetermined range around the biodegradability, for instance, by determining whether a difference between the determined biodegradability and the target biodegradability lies below a predetermined threshold. However, the comparison can also refer to a more complex mathematical function and the condition for which the potential target formulation is determined as the target formulation can refer to any condition that is based on the comparing of the determined biodegradability with the target biodegradability. Generally, if the predetermined condition is fulfilled, for instance, if the determined biodegradability lies within the predetermined range around the target biodegradability, the iteration control unit 116 determines that the potential target formulation is the target formulation and that the potential target preparation specification is the target preparation specification and ends the iteration.

If the above condition is not fulfilled, for instance, if the determined biodegradability lies not within the predetermined range around the target biodegradability, the iteration control unit 116 is adapted to decide that a further iteration step is necessary. In this case, the iteration control unit 116 is adapted to provide a new potential target preparation specification of a potential target formulation and to repeat the determination of the biodegradability utilizing the new potential target preparation specification of the new potential target formulation. For example, the new potential target formulation preparation specification can be provided on a database on which a plurality of potential target preparation specifications are already stored and from which the iteration control unit 116 can select a new potential target preparation specification arbitrarily or according to predetermined rules. Such rules can, for instance, be a function of the comparison of the determined biodegradability with the target biodegradability of the potential target formulation. For example, the function can refer to the size of the difference between the determined biodegradability and the target biodegradability, wherein the smaller the difference the more parts of the new potential target formulation are similar in the potential target formulation. In such a case, these rules can lead to the iteration control unit 116 being adapted to select new potential target formulations that are more similar to the potential target formulation if the determined biodegradability for the potential target formulation is already similar to the target formulation and that are less similar if the difference between the determined biodegradability and the target biodegradability is high. However, also completely different rules can be applied. Moreover, the iteration control unit 116 can also be adapted to generate a new potential target preparation specification, for instance, based on the potential target preparation specification and predetermined rules or arbitrarily. Also in this case for the rules the same principles as described above can be applied.

Moreover, the iteration control unit 116 can also be adapted to apply an abortion criterion for the iteration that indicates that for a respective target biodegradability no suitable target preparation specification can be found. For example, the iteration control unit 116 can be adapted to apply an abortion criterion that refers to a predetermined number of iteration steps, i.e. that refers to determine a predetermined number of new potential target preparation specifications. However, also other abortion criteria can be utilized.

An output unit referring, for instance, to a display, can then be adapted to output the determined target preparation specification or the target formulation, for instance, in form of a visual representation of the formulation, an identification of the formulation, a chemical formula representing the formulation, components and quantities of the formulation, etc. Moreover, the output unit can additionally or alternatively be adapted to provide the determined target preparation specification to a database 140 for storing the respective determined target preparation specification in association with the respective target biodegradability for a future usage. Optionally, the apparatus 110 can comprise the control unit 117 that is adapted to provide control signals based on the determined target preparation specification for controlling a production process of the production system 120. In particular, it is preferred that the control signals are indicative of the machine executable preparation specification of the target formulation which is generated based on the determined target preparation specification for producing the target formulation fulfilling the target biodegradability. However, the control unit 117 can also be adapted to control the production process of another product based on the determined target preparation specification, for instance, to provide control signals indicative of a machine executable preparation specification for another product utilizing or comprising the respective target formulation.

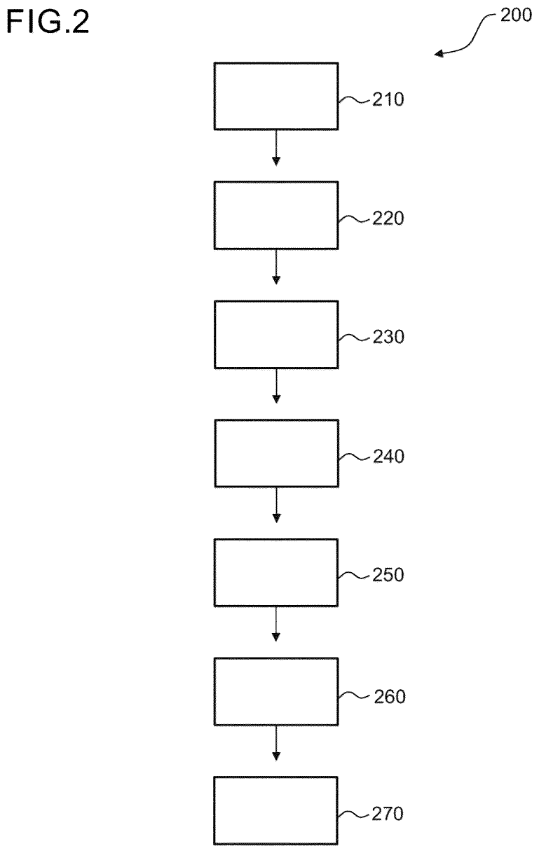

FIG. 2 shows schematically and exemplarily a flow chart of a method for determining a target preparation specification indicative of a target formulation comprising a target biodegradability. The method 200 comprises a first step 210 of providing a target biodegradability. Further, in a step 220 a digital representation of a potential target preparation specification of a potential target formulation are provided. In particular, the providing of the target biodegradability and of the digital representation can be in accordance with the principles described above with respect to the target biodegradability providing unit 111 and the digital representation providing unit 112, respectively. Further, in a step 230 a biodegradation habitat indicative of habitat descriptor values of habitat descriptors influencing a biodegradation of a formulation in a respective habitat is provided. Also for this step 230, the principles described above, for instance, with respect to the habitat providing unit 113 can be applied. Further, in step 240 a biodegradation model is provided that is adapted to determine the biodegradability of the formulation based on the digital representation. As already discussed above in more detail, the providing of the biodegradation model can also refer to a selection of the biodegradation model based on the provided biodegradation habitat. Moreover, the biodegradation model is a data driven model parameterized with respect to the biodegradation habitat such that it can determine a biodegradability of a formulation, preferably, based on the characterizing parameters of the formulation. Generally, the steps 210, 220, 230 and 240 can be performed in arbitrary order or even concurrently. In a following step 250 a biodegradability is determined based on the provided digital representation of the potential target formulation and the biodegradation model. In a step 260 the determined biodegradability of the potential target formulation is then compared with the target biodegradability. Based on this comparison, either the potential target formulation is determined as a target formulation and the potential target preparation specification as a target preparation specification, or a new potential target preparation specification of a new potential target formulation is provided and the determination of the biodegradability utilizing the new potential target preparation specification of the potential target formulation is repeated. In an optional step 270 after a target preparation specification has been determined utilizing the steps above, the determined target preparation specification together with the determined target formulation and the target biodegradability can be provided to a user via an output unit. Moreover, in the step 270 the potential target preparation specification can also be utilized for generating control signals that allow for a controlling of a production process of a product, for instance, of the target formulation or of a product comprising the target formulation, as already described above in detail.

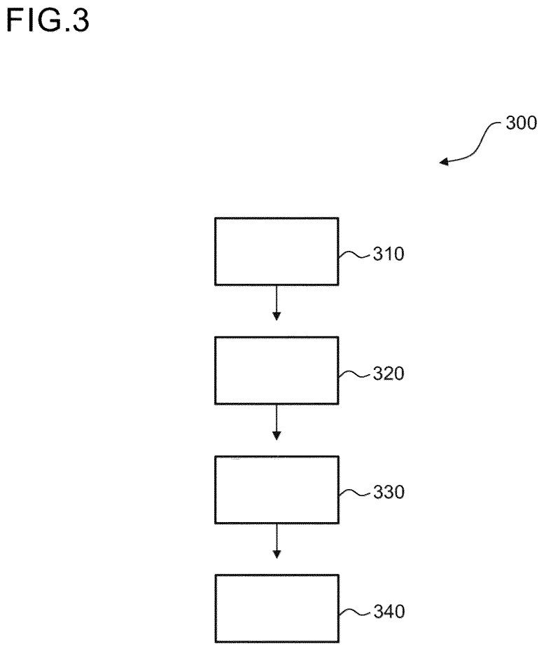

FIG. 3 shows schematically and exemplarily a flow chart of a method for training the data driven based biodegradation model utilized, for instance, in the method 200 discussed with respect to FIG. 2. Generally, the method 300 can be perform, for instance, by respective units of the training apparatus 130 as described with respect to FIG. 1. The method 300 comprises a step 310 of providing training data for training the data driven based biodegradation model. The training data comprises a) a digital representation of a plurality of training formulations, and b) a biodegradability associated with each training formulation in a respective biodegradation habitat, for instance, for specific habitat descriptor values. Optionally, the training data set can further comprise the respective specific habitat descriptor values. In particular, the training data can be provided in accordance with the principles described above with respect to the training data providing unit 131 described with respect to FIG. 1. The method comprises further a step 320 of providing a data driven based trainable biodegradation model, for instance, a machine learning based biodegradation model like a neural network. Generally, the step 310 and the step 320 can be performed in arbitrary order or even at the same time. The method 300 then further comprises a step 330 of training the provided data driven based biodegradation model based on the provided training data, for instance, by varying parameters in the data driven based trainable biodegradation model, such that the trained biodegradation model is adapted to determine a biodegradability of a formulation based on a digital representation of the formulation. In step 340 the trained biodegradation model can then be provided, for instance, by storing the trained biodegradation model on a storage or by directly providing the trained biodegradation model to the apparatus 130 as described with respect to FIG. 1.

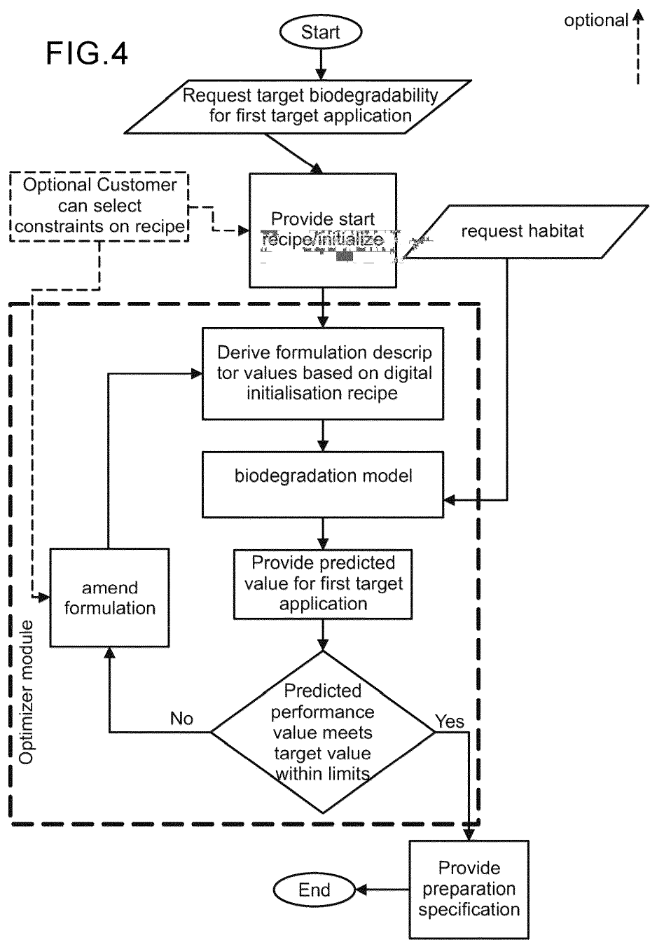

In the following, more detailed preferred examples of the above described method and the corresponding apparatus will be described. A schematic and exemplary flow chart of an exemplary and preferred embodiment of the method is provided by FIG. 4. In this exemplary embodiment, the method starts with requesting, for instance, via a user interface, a target value for a target application, in particular, a target biodegradability. Moreover, in a next step, the optimization is initialized by providing a potential target preparation specification, i.e. a start recipe. Optionally, constraints on the recipe, i.e. the preparation specification, can be taken into account in this process, for instance, if a user provides such constraints. The constraints can refer, for instance, to constraints in the production of a formulation, in the starting substances that should be used for synthesizing the formulation, etc. Moreover, additional application conditions can be requested being in particular indicative of the biodegradation habitat for the target formulation. Moreover, additional application conditions can also be indicative of further information with respect to the target formulation that should be fulfilled. For example, the requested additional application conditions can refer to a geolocation indicating where it is expected that the formulation might biodegrade, wherein based on these geolocations the biodegradation habitat and the respective habitat descriptors can be determined, for instance, by utilizing a database on which respective associated biodegradation habitats and biodegradation physicochemical parameters are already stored. Based on the above steps, the optimization for determining the target formulation, i.e. the target preparation specification, can be initialized. In a first step of the optimization, characterizing parameter values can be derived from the provided start recipe, i.e. from the provided potential target preparation specification. However, the deriving of the characterizing parameters can also refer to accessing a storage on which respective characterizing parameter values for the respective potential target formulation are already stored. Moreover, if the provided digital representation of the potential target preparation specification already comprises the characterizing parameters, this step can also be omit-ted. Based on the requested additional application conditions, in particular, based on the biodegradation habitat, a respective determination model, i.e. a biodegradation model, can be provided. Based on the provided determination model and the digital representation of the potential target preparation specification, a value for the target application, i.e. the biodegradability, for the potential target formulation can be provided. In a next step it is determined if the determined performance value, i.e. the determined biodegradability, meets the target value, i.e. the target biodegradability, within predetermined limits. If this is not the case, i.e. if this condition is not fulfilled, the formulation of the potential target preparation specification is amended and a new potential target preparation specification is determined optionally taking into account the constraints previously provided. The iteration can then start anew for the new potential target preparation specification. If at one point the determined performance value meets the target value within limits, i.e. if the respective condition is fulfilled, the potential target preparation specification is determined as the target preparation specification and provided, for example, to a user or to a control unit for producing the respective determined target formulation.

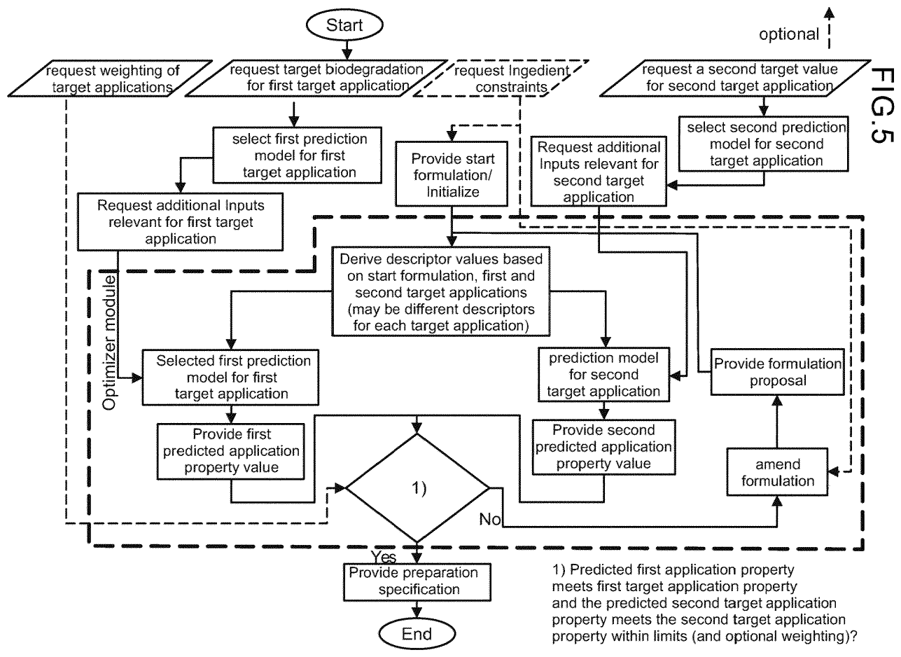

FIG. 5 shows schematically and exemplarily a further preferred embodiment of the above method for determining a target preparation specification with predetermined target biodegradability, wherein in this embodiment in addition to the target biodegradability it is desired that the target formulation also fulfills a further target value, i.e. target technical application property. The additional target technical application property can refer to any technical application property, for instance, also to an additional biodegradability in another habitat, or any other technical application property. Generally, the method follows the same principles as described above with respect to FIG. 4. However, due to the additional target value, additional conditions have to be met during the optimization. Thus, in the following only the main differences with respect to the method as described above will be pointed out. In particular, in this preferred embodiment, the optimizer module does not only optimize over the first target value, i.e. over the target biodegradability, but also over the second target value. Preferably, also for the second target value a determination model adapted for determining a value for the technical application property based on characterizing parameters is utilized. Thus, in addition to the method as described above for the second target application a second determination model is provided that allows to determine an application property value based on the characterizing parameters for the second target application. The second determination model can, for instance, be based on the same algorithm as the biodegradation model, and is only trained with a different data set such that it determines another property of the formulation. The comparison then refers to not only determining whether the determined biodegradability meets the target biodegradability within limits, but also whether the determined second application property value meets the target second application property value within limits. Predetermined rules can be utilized that determine for which cases the iteration is continued, i.e. a new formulation is provided as new potential target preparation specification and for which conditions the potential target preparation specification is determined as the target preparation specification. For example, a user can predetermine weights for weighting to which extents which of the conditions has to be met. For instance, it can be more important for a user that the biodegradability is met, whereas the other target application property is not so important. In this case, either the limits within which the second target application property can be met can be set broader or the meeting of this condition can be weighted less strongly. In this context also Pareto optimization methods can be utilized to find an optimal trade of between the different targets. If at one point of the iteration it is then determined that the conditions are met and fulfil the predetermined rules the respective potential target preparation specification can be determined as target preparation specification and provided as output to a user or can be utilized to generate a control file for producing the respective target formulation.

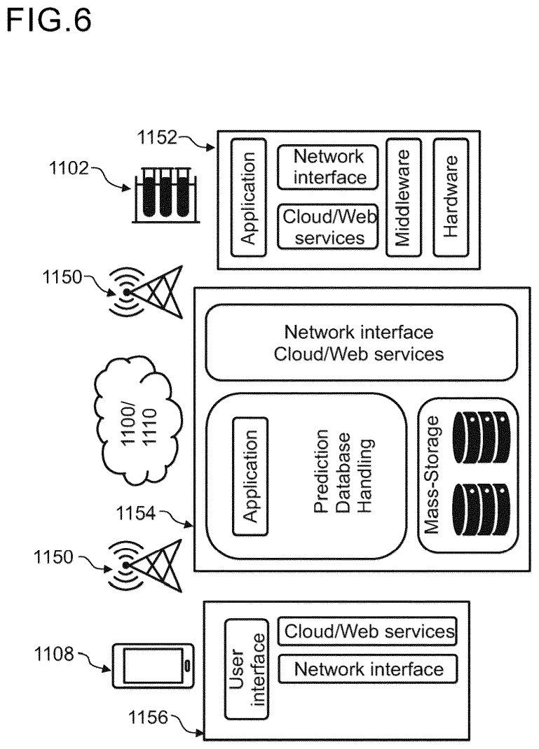

FIG. 6 illustrates a block diagram of an exemplarily system architecture of an automated laboratory system 1000 for preparing a formulation with a laboratory equipment control device 1102, a network 1150 and the preparation specification, i.e. recipe, module 1100/1110, and a client device 1108. The automated laboratory system includes a laboratory equipment control device layer 1152 as part of the laboratory equipment control device 1102 as well as a preparation specification module layer 1154 associated with the preparation specification module and a remote control or client layer 1156 associated with the client device 1108. The laboratory equipment control device layer can be split into several hierarchical layers: the hardware, the middleware and the interface layer. The hardware layer relates to hardware resources such as sensors and actuators, in particular for controlling a preparation of a formulation. The middleware relates to any of the known middle-ware for laboratory or plant preparation operations. One example is LABS/QM, providing different abstractions to hardware, network and operating system such as low-level device control and message passing. The communication layer relates to communication protocols, wherein one of the protocol may be REST, which may be implemented over different transport protocols (i.e. UDP, TCP, Telemetry) that allow the exchange of messages between the laboratory equipment control device and laboratory equipment devices. Such software architecture allows to control and monitor laboratory equipment without having to interact with the hardware.

The preparation specification module layer 1154 may include: a mass storage layer, the computing layer, the interface layer. The storage layer is configured to provide mass storage for the data-driven biodegradation model for providing a recipe, i.e. preparation specification, of a formulation that meets a target biodegradability, as described in detail above. In particular, the functions performed by the apparatus, as described above, can be provided as program code means stored on the mass storage. Furthermore, preparation specifications for a plurality of formulations can be stored in the mass storage. Such data may be stored in structured databases such as SQL databases or in a distributed file system such as HDFS, NoSQL databases such as HBase, MongoDB. The computing layer may include an application layer that allows to customize the functionalities provided by standard cloud services to perform computing processes based on target properties. Such functionalities can include determining based on a target biodegradability and the biodegradation model a digital representation of a target formulation, generating a preparation specification from the digital representation of the target formulation, and providing the preparation specification as control data to the laboratory equipment control device.

The interface layer may implement web services, network interfaces as UDP or TCP or Websocket interfaces. For communication with the laboratory equipment control device a REST API is implemented.

The client layer 1156 provides interfaces for end-users. For end-users, the client layer 1156 can run client side Web applications, which provide interfaces to the preparation specification module layer 1154 or the laboratory equipment control device layer 1152. Users may be provided with a UI for selecting a target biodegradability and a biodegradation habitat for the target biodegradability, the target biodegradability may also comprise a range of biodegradability values. In other examples, the users may be provided with a UI for selecting more than one target biodegradability and respective values. The applications may be configured for users to monitor and control the laboratory equipment control device and the operation remotely. In other examples, the client device layer and the preparation specification module layer may be integrated into one device. The alternatives described here are only for illustration purposes and should not be considered limiting.

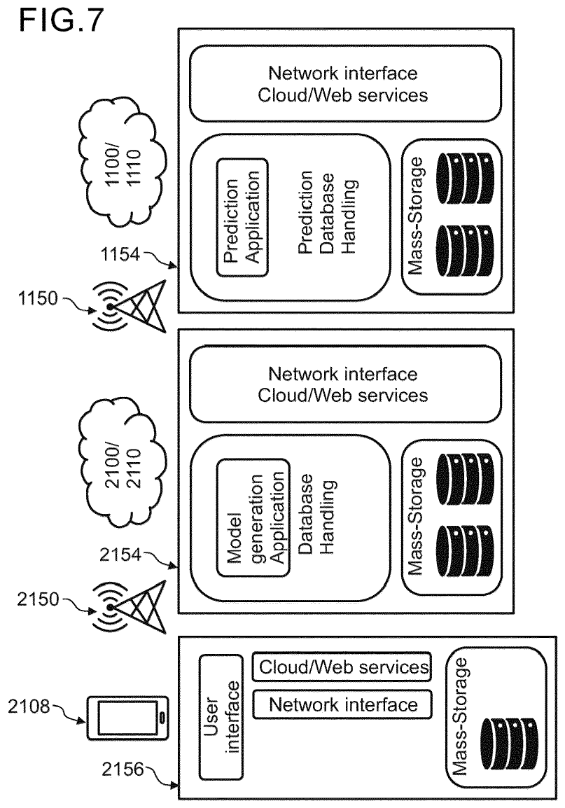

FIG. 7 illustrates a block diagram of an exemplarily system architecture of a system and apparatus for generating a biodegradation model for determining a biodegradability, a network 2150 and a model generating module 2100/2110 that can be regarded as or comprising a training model apparatus, a preparation specification module 1100/1110, and a client device 2108. The system for generating a biodegradation model includes a model generating module layer 2154 as part of a model generating module and a client layer 2156 associated with the client devices 2108.

The model generating module layer 2154 may include: a mass storage layer, a computing layer, an interface layer. The storage layer is configured to provide mass storage for the data-driven biodegradation model as described above. Furthermore, the mass storage is configured for storing preparation specifications for formulations and measured biodegradabilities for one or more habitats. Such data may be stored in structured databases such as SQL databases or in a distributed file system such as HDFS, NoSQL databases such as HBase, MongoDB. The computing layer may include an application layer that allows to customize the functionalities provided by standard cloud services to perform computing processes for generating a biodegradation model for determining a biodegradability of a formulation. Such functionalities may include receiving for at least two previously measured formulations their respective digital representations associated with a preparation specification, measurement data of at least one biodegradability in at least one habitat for each of the at least two previously measured formulations, receiving at the model generating module the digital representation of at least one unmeasured formulation, training the model according to the above described training principles based on the digital representation of the at least two previously measured formulations, the measurement data of the biodegradability in the at least one habitat for each of the at least two previously measured formulations, and, preferably, a similarity measure between the digital representation associated with the preparation specification of each of the at least two previously measured formulations and the respective digital representation associated with a preparation specification of the at least one unmeasured formulation, and providing via an output interface the biodegradation model for the biodegradability. The model generating module layer may be configured for deploying the generated model and the preparation specification database to the preparation specification module layer. This may include storing the generated model and the preparation specification database in the mass storage devices associated with the preparation specification module.

The model generating module layer may further be configured for determining a digital representation of the formulation associated with the preparation specification from the preparation specification. The digital representation may include a set of characterizing parameters associated with a preparation specification of each measured formulation. One way of deriving these characterizing parameters can be to apply the SMILES algorithm or any other already above described principle. In case, where the model is generated based on the digital representation derived from the recipe, a relation between the preparation specification and the characterizing parameters may be stored in the mass storage devices associated with the model generating module. In such cases, deploying the model comprises providing that relation.

The interface layer may implement web services, network interfaces as UDP or TCP or Websocket interfaces. For communication with the client device a REST API is implemented in this example. The client layer 2156 provides access to mass storage devices, that contain preparation specifications for formulations, and for at least two formulations at least one biodegradability. The client layer further provides an interface for end-users. For end-users, the client layer 2156 may run client side Web applications, which provide interfaces to the model generation module layer 2154 or the mass storage devices associated with the client layer. Users may be provided with a UI for selecting a test method and/or habitat for which the biodegradability shall be determined. The user may further be provided with a UI for selection of the preparation specification data. The user interface may also provide an option for uploading the selected data to the model generating module layer and optionally an option to initiate model generation.

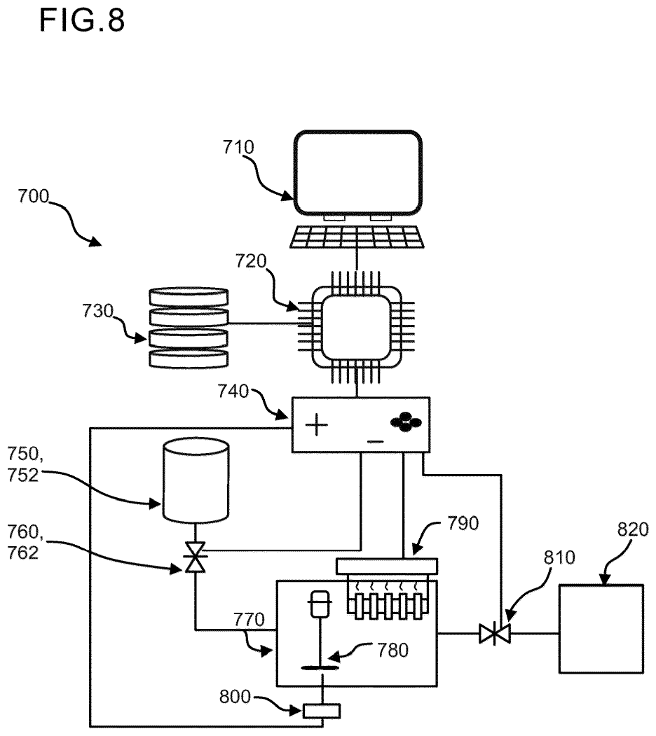

FIG. 8 shows an exemplary system 700 for producing a chemical product based on a preparation specification generated according to the invention. In this example the system comprises a user interface 710 and a processor 720, associated with a control unit 740. The user interface 710 and the processor 720 can be associated with or realized in accordance with the principles described above, in particular, can be adapted to perform a computer implemented method to determine a target formulation and/or preparation specification based on a determined biodegradability, as described above. The control unit 740 is, for example, configured for receiving control data generated according to the invention as described above, in particular, to receiving control data generated based on a preparation specification of a formulation comprising a target biodegradability. In this example the control data is provided from a data base 730, in other examples, however the control data can also be provided from a server or any other computational unit for distributing data. Vessels 750, 752 each contain a component of the chemical product, for example, components, catalysts, etc. In general more than two vessels are present, however, in this example for illustrative purposes only two are shown. Valves 760, 762 are associated with vessels 750, 752. Valves 750 and 752 can be controlled to dose appropriate amounts of each component into reactor 770, according to the preparation specification. A motor 800 of a mixer 780 may also be controlled by the control unit according to the preparation specification. An optional heater 790 may also be controlled according to the preparation specification. Finally, an exit valve 810 in fluid communication with the reactor may be controlled by the control unit to provide the chemical product to a container or test system 820.

In the following a more detailed example of a biodegradation model is described. In this example the biodegradation model is trained based on a training data set comprising hundred or more data points comprising respective formulations including composition and biodegradation, for instance, according to a certain norm, e.g. OECD 301a-f. The composition of a formulation can be defined utilizing IDs of the chemical components of the formulation and their amounts. For training or applying the biodegradation model chemical components of a formulation can then be sorted in predetermined or learned component classes. For example, the classes can comprise solvents, surfactants, pigments, etc. For each component of a class predetermined descriptors can be provided, for example, stored on a respective database or derived, as described above in more detail. The descriptors can be partition coefficients, functional group counts, pKa values, molar mass distribution, glass transition temperatures, etc. Also, a biodegradability according to a certain norm (e.g. OECD 301a-f) of individual components can be used as descriptor. Also computed descriptors like computed polarities, partition coefficients, critical micelle concentrations, solubilities, etc. can be utilized. Further, if the chemical structure of the components is known also molecular fingerprints, like Morgan fingerprints, can be used as descriptors. The descriptors of a respective class can be standardized, for instance, with a z-score normalization, within the component class. Moreover, if water is a used component in a formulation, this component can be removed from the component list and the remaining components can be normed to 100 weight %. Then, for each descriptor a descriptor value can be derived for all components from a component class of a given formulation, for instance as an average descriptor value or a minimal or maximal descriptor value. For example, an averaging can refer to using the weight fraction of an individual component in the formulation without water as a weighting factor. In case of Morgan count fingerprints, a sum can be taken of the weight %-weighted fingerprints of the individual components of a component class. A total amount of weight fraction within each component class can also be used as descriptor. Moreover, in addition to the descriptors computed properties of the formulation can be additionally used as input for the biodegradation model, for example viscosity, solid content, etc. Based on these input parameters of a formulation a Random Forest algorithm as biodegradation model can be trained to determine the amount of biodegradation in a habitat, for example, according to a certain norm, e.g. OECD 301a-f. The biodegradation model can be parameterized based on a respective training data set. Further a respective test data set can used for testing and validating the trained biodegradation model. The data for the training data set and the test data set can be acquired by measuring a biodegradation of respective formulations in a respective habitat in a comparable way, e.g. according to norm OECD 301a-f. Moreover, also already existing databases, published data sets or literature can be utilized. One example for published biodegradation data for polymer blends can be found, for example, in “Blends of PBAT with plasticized starch for packaging applications: mechanical properties, rheological behavior can biodegradability” M. Dammak, et al., Industrial Crops & Products, 144, 112061 (2020).

In the following some further details with respect to some of the above described embodiments are provided. Generally, in some applications it is desirable to find a formulation that meets a certain technical application property, like tensile strength and also meets a requirement regarding biodegradability. For this application a method is proposed, for instance, as described with respect to FIG. 5. In an example for this embodiment suitable for this application, a target requirement for the biodegradability can be provided, and further a target application property is provided. Based on the target application property a determination model is selected, wherein the determination model relates preferably characterizing parameters associated with a preparation specification to an application property. In addition, a further model is selected based on the habitat of the formulation. This biodegradation model relates preferably habitat information and characterizing parameters associated with a preparation specification to a biodegradability. Based on the target application requirements characterizing parameters based on the preparation specification are determined. In an optional step additional descriptor values for the habitat are requested based on the used selected biodegradation model. Based on the biodegradation model the biodegradability can be determined. The determined biodegradability is then compared with the target biodegradability. Further, the determination model is used for determining the application property of the formulation and the determined application property is compared to the target property. In case the determined biodegradability meets the target biodegradability and the determined application property meets the target property, the preparation specification of the formulation is provided. The preparation specification can also refer or include control data for controlling a plant for producing the formulation. In case the target biodegradability and/or the target application property is not met, the target application property can be reduced and the process reruns with a reduced target application requirement, until the biodegradation requirement can be met. An acceptable range of the target application property may be provided. If no formulation is found that meets the required targets of biodegradability and target application performance, the process can stop and the user can be notified.

Potential representations of the biodegradability may be one or more of a mineralization referring to information whether the formulation fully mineralizes or not or a time until the mineralization is achieved, a biotransformation referring to an alteration in the chemical structure resulting in the loss of a specific property of the formulation, e.g. toxicology, or time until this is achieved, a half-life referring to the time until 50% of the formulation are decomposed. Prominent habitats are Marine, waste water, and soil. For marine the following parameters can have an influence on the biodegradation: salt concentration, sediments, water temperature, bacterial cultures, etc. In some examples, the marine habitat descriptors can be stored in a database together with the geolocation. In that case the geolocation can be entered and the values of the parameters related to this geolocation can be retrieved from a database. For waste water, the following parameters can have an influence on biodegradation: Temperature, bacteria population, bacteria type, enzyme concentration, enzymes. For soil the following parameters can have an influence on biodegradation, temperature, bacteria population, bacteria type, enzyme concentration, enzymes.

Other variations to the disclosed embodiments can be understood and effected by those skilled in the art in practicing the claimed invention, from a study of the drawings, the disclosure, and the appended claims.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the providing of the digital representation and the biodegradation model, the determining of the biodegradability, the providing of the biodegradability, etc. performed by one or several units or devices can be performed by any other number of units or devices. These procedures can be implemented as program code means of a computer program and/or as dedicated hardware.

A computer program product may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would under-stand that the structure of an executable component may include software objects, routines, methods, and so forth, that may be executed on the computing system. This may include both an executable component in the heap of a computing system, or on computer-readable storage media. The structure of the executable component may exist on a computer-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other in-stances, structures may be hard coded or hard wired logic gates, that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communications connection, for example, either hardwired, wireless, or a combination of hardwired or wireless, to a computing system, the computing system properly views the connection as a transmission medium. Transmission media can include a network and/or data links which can be used to carry desired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, main-frame computers, mobile telephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

The invention refers to a method for determining a preparation specification comprising a target biodegradability. A target biodegradability is provided indicative of a biodegradation characteristic of a formulation. A digital representation of a potential target preparation specification is provided indicative of physicochemical characteristics of the formulation. A habitat is provided indicative of habitat descriptor values of habitat descriptors. A model is provided based on the habitat that is adapted to determine the biodegradability of a formulation in the habitat. The biodegradability of the potential target formulation is determined based on the provided model and the digital representation. Then the determined biodegradability is compared with the target biodegradability and either i) the potential target formulation is determined as the target formulation, or ii) a new potential target preparation specification of a potential target formulation is provided and the determination of the biodegradability is repeated utilizing the new potential target preparation specification.

1. A computer implemented method for determining a target preparation specification indicative of a target formulation comprising a target biodegradability, wherein the method comprises:
providing a target biodegradability, wherein a biodegradability is indicative of a biodegradation characteristic of a potential target formulation,
providing a digital representation of a potential target preparation specification of the potential target formulation,
providing a biodegradation habitat, wherein the biodegradation habitat is indicative of habitat descriptor values of habitat descriptors influencing a biodegradation of a formulation in the respective habitat, wherein the habitat descriptors are indicative of environmental characteristics of the habitat,
providing biodegradation model based on the provided biodegradation habitat, wherein the biodegradation model is adapted to determine the biodegradability of a formulation in the respective biodegradation habitat, wherein the biodegradation model is a data driven model parameterized with respect to the biodegradation habitat such that it determines a biodegradability of a formulation based on a digital representation of the formulation,
determining the biodegradability of the potential target formulation based on the provided biodegradation model and the digital representation, and
comparing the determined biodegradability of the potential target formulation with the target biodegradability and, based on the comparison, either i) determining the potential target formulation as the target formulation and the potential target preparation specification as the target preparation specification, or ii) providing a new potential target preparation specification of a potential target formulation and repeating the determination of the biodegradability utilizing the new potential target preparation specification of the potential target formulation.

2. The method according to claim 1, further comprising providing a target technical application property for the target formulation and providing the potential target preparation specification based on the provided target technical application property such that the potential target formulation fulfils the provided target technical application property.

3. The method according to claim 2, wherein the providing of a new potential target preparation specification is based on amending the provided target application property and providing the new potential target preparation specification such that the potential target formulation fulfils the amended target application property.

4. The method according to claim 2, wherein the providing of the potential target preparation specification based on the provided target technical application property comprises utilizing a determination model adapted to determine a technical application property of a formulation based on the digital representation of the formulation, wherein the determination model is a data driven model parameterized such that it determines based on the digital representation of the formulation the technical application property associated with the formulation.

5. The method according to claim 1, wherein the providing of the target preparation specification of the target formulation comprises providing control signals adapted for controlling an industrial plant for producing the target formulation in accordance with the target preparation specification.

6. The method according to claim 1, wherein the biodegradation habitat refers to any one of a marine habitat, a waste water habitat, a limnic habitat, a compost habitat, an anaerobic habitat or a soil habitat.

7. The method according to claim 6, wherein the biodegradation habitat refers to a marine habitat and wherein the habitat descriptors refer to at least one of a salt concentration, a sedimentation type, oxygen level, location, sample depth, a water temperature, a nutrient concentration, a pH value, an environmental type and a microbial community.

8. The method according to claim 6, wherein the biodegradation habitat refers to soil and the habitat descriptors refer to at least one of a temperature, a sand content, a pH value, a moisture content, a nutrient concentration, a microbial community and an enzyme environment.

9. The method according to claim 1, wherein habitat descriptor values for the habitat descriptors are stored associated with respective geolocations, wherein the providing of a biodegradation habitat refers to providing a geolocation of the habitat and retrieving the habitat descriptor values for the geolocation from storage.

10. An interface method for providing an interface, wherein the interface method comprises:
receiving as input a target biodegradability, start digital representation and a habitat via a user interface and providing the received target biodegradability, digital representation and the habitat to a processor performing the method according to claim 1, and
providing the target preparation specification of the formulation as result, wherein the result is received from the processor performing the method according to claim 1.

11. A computer implemented training method for training a data driven based biodegradation model for parameterizing the biodegradation model, wherein the training method comprises:
providing training data associated with a predetermined biodegradation habitat, wherein the training data comprises a) digital representations of a plurality of training formulations, and b) a biodegradability for the respective biodegradation habitat associated with each training formulation,
providing a data driven based trainable biodegradation model,
training the provided data driven based biodegradation model based on the provided training data such that the trained biodegradation model is adapted to determine a biodegradation of a formulation based on the digital representation of the formulation, and
providing the trained biodegradation model.

12. An apparatus for determining a target preparation specification indicative of a target formulation comprising a target biodegradability, wherein the apparatus comprises:
a target biodegradability providing unit for providing a target biodegradability, wherein a biodegradability is indicative of a biodegradation characteristic of a potential target formulation,
a digital representation providing unit for providing a digital representation of a potential target preparation specification of the potential target formulation,
a habitat providing unit for providing a biodegradation habitat, wherein the biodegradation habitat is indicative of habitat descriptor values of habitat descriptors influencing
a biodegradation of a formulation in the respective habitat, wherein the habitat descriptors are indicative of environmental characteristics of the habitat,
a model providing unit for providing a biodegradation model based on the provided biodegradation habitat, wherein the biodegradation model is adapted to determine the biodegradability of a formulation in the respective biodegradation habitat, wherein the biodegradation model is a data driven model parameterized with respect to the biodegradation habitat such that it determines a biodegradability of a formulation based on the digital representation of the formulation,
a biodegradability determination unit for determining the biodegradability of the potential target formulation based on the selected biodegradation model and the digital representation, and
an iteration control unit for comparing the determined biodegradability of the potential target formulation with the target biodegradability and, based on the comparison, either i) determining the potential target formulation as the target formulation and the potential target preparation specification as the target preparation specification, or ii) providing a new potential target preparation specification of a potential target formulation and repeating the determination of the biodegradability utilizing the new potential target preparation specification of the potential target formulation.

13. An interface apparatus for providing an interface, wherein the interface apparatus comprises:
an input interface unit for receiving as input a target biodegradability, a start digital representation and a habitat via a user interface and for providing the received target biodegradability, start digital representation and the habitat to an apparatus according to claim 12, and
an result interface for providing the habitat descriptor values of the formulation as result, wherein the result is received from the apparatus according to claim 12.

14. A training apparatus for training a data driven based biodegradation model for parameterizing the biodegradation model, wherein the training apparatus comprises:
a training data providing unit for providing training data associated with a predetermined biodegradation habitat, wherein the training data comprises a) digital representations of a plurality of training formulations, and b) a biodegradability for the respective biodegradation habitat associated with each training formulation,
a trainable model providing unit for providing a data driven based trainable biodegradation model,
a training unit for training the provided data driven based biodegradation model based on the provided training data such that the trained biodegradation model is adapted to determine a biodegradation of a formulation based on the digital representation of the formulation, and
a trained model providing unit for providing the trained biodegradation model.

15. A computer program product for determining a target formulation comprising a target biodegradability, wherein the computer program product comprises program code means for causing the apparatus of claim 12 to execute the method according to claim 1.
